SEQUENCE LISTING (16 sequences)

SEQ ID NO: 1 - DNA, 690 bp, Vesicular stomatitis virus
atgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatga

SEQ ID NO: 2 - DNA, 1139 bp, Artificial Sequence
  The nucleotide sequence of Entranceposon, a vector with the function of transposition
gatctgcggccgcgcacgaaaaacgcgaaagcgtttcacgataaatgcgaaaacggatcgatcctagtaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaactggatcatccgttttcgcatttatcgtgaaacgctttcgcgtttttcgtgcgcggccgcagatc

SEQ ID NO: 3 - DNA, 690 bp, Vesicular stomatitis virus
atgagttccttaaagaagattctcggtctgaaggggaaaggtaagaaatctaagaaattagagatcgcaccacccccttatgaagaggacactagcatggagtatgctccgagcgctccaattgacaaatcctattttggagttgacgaggcggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggctttcgcaggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtggttcctggactctatcggccacttcaaatga

SEQ ID NO: 4 - DNA, 13513 bp, Artificial Sequence
  The nucleotide sequence of pRV-core Mut4 vector
ttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcatacttctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactataggacgaagacaaacaaaccattattatcattaaaaggctcaggagaaactttaacagtaatcaaaatgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagagatcgcaccacccccttatgaagaggacactagcatggagtatgctccgagcgctccaattgacaaatcctattttggagttgacgaggcggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttgcaggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtggttcctggactctatcggccacttcaaatgagctagtctaacttctagcttctgaacaatccccggtttactcagtctcccctaattccagcctctcgaacaactaatatcctgtcttttctatccctatgaaaaaaactaacagagatcgatctgtttacgcgtcactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgctaggtatgaaaaaaactaacagatatcacgctcgagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctagccagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacgtcatgtcaagccaatcccatcccaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcggatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagtgcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgt

SEQ ID NO: 5 - DNA, 74 bp, Artificial Sequence
  The nucleotide sequence of 2MCS
ctcgagcgccaccggcgcgcctctagctatagcatgaaaaaaactaaacagatatcatggctaagcgcggccgc

SEQ ID NO: 6 - DNA, 14359 bp, Artificial Sequence
  The nucleotide sequence of pRV-2MCS vector
taatacgactcactataggacgaagacaaacaaaccattattatcattaaaaggctcaggagaaactttaacagtaatcaaaatgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatgagctagtctaacttctagcttctgaacaatccccggtttactcagtctcccctaattccagcctctcgaacaactaatatcctgtcttttctatccctatgaaaaaaactaacagagatcgatctgtttacgcgtcactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgctaggtatgaaaaaaactaacagatatcacgctcgagcgccaccggcgcgcctctagctatagcatgaaaaaaactaaacagatatcatggctaagcgcggccgccagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacgtcatgtcaagccaatcccatcccaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcggatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagtgcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgtgggtcggcatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataactagcataaccccttggggcctctaaacgggtcttgaggggttttttgctgaaaggaggaactatatccggatcgagacctcgatactagtgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcatacttctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattg

SEQ ID NO: 7 - DNA, 6058 bp, Artificial Sequence
  The nucleotide sequence of a plasmid comprising the N gene in VSV core backbone
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcatgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgactcgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttacgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 8 - DNA, 11119 bp, Artificial Sequence
  The nucleotide sequence of a plasmid comprising the L gene in VSV core backbone
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcatggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacgtcatgtcaagccaatcccatcccaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcggatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagtgcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaactcgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttacgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 9 - DNA, 5587 bp, Artificial Sequence
  The nucleotide sequence of a plasmid comprising the P gene in VSV core backbone
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagctcgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttacgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 10 - DNA, 1407 bp, Artificial Sequence
  The nucleotide sequence of the heavy chain of the anti-PDL1 antibody
atggagacagacacactcctgctatgggtactgctgctctgggttccaggttccactggtgaggtgcagctggtggaatcaggaggaggactggtgcagccaggaggatctctgagactgtcttgcgccgccagcggctttacattcagcgactcttggatccattgggtgcgccaggctccaggaaaaggactggagtgggtggcttggatcagcccttacggcggcagcacctactacgccgatagcgtgaagggcaggttcaccatcagcgccgataccagcaagaacaccgcctacctgcagatgaacagcctgagggccgaggataccgccgtgtactattgcgccaggaggcattggccaggcggatttgactattggggccagggaacactggtgacagtgtcagccgccagcaccaagggacctagcgtgtttcctctggcccctagcagcaagtctacaagcggcggcacagccgctctgggttgtctggtgaaggactacttccccgagccagtgaccgtgtcttggaacagcggagctctgacaagcggagtgcacacatttccagccgtgctgcagagctcaggactgtacagcctgtccagcgtggtgacagtgccttctagcagcctgggcacccagacctacatctgcaacgtgaaccacaagcccagcaacaccaaggtggacaagcgggtggagcccaagtcttgcgacaagacccacacttgccccccttgtccagctccagaactgctgggaggaccatcagtgttcctgttcccccctaagcccaaggacaccctgatgatcagccggaccccagaagtgacttgcgtggtggtggacgtgtctcacgaggaccccgaggtcaagttcaattggtacgtggacggagtggaggtgcacaacgctaagaccaagcccagggaggagcagtacaacagcacctacagggtggtgtccgtgctgacagtgctgcaccaggattggctgaacggcaaggagtacaagtgcaaggtgtccaacaaggccctgccagctcccatcgagaagaccatcagcaaggccaagggccagcctagagagcctcaggtgtacacactgccccctagcagagaggagatgaccaagaaccaggtgtccctgacttgcctcgtgaagggcttctaccccagcgatatcgccgtcgagtgggaatctaacggccagcccgagaacaactacaagaccaccccccccgtgctggatagcgacggcagcttcttcctgtacagcaagctgaccgtggacaaaagtcgctggcagcagggcaacgtgttctcttgcagcgtgatgcacgaggccctgcacaaccactacacccagaagagcctgagcctgagcccaggaaagtag

SEQ ID NO: 11 - DNA, 705 bp, Artificial Sequence
  The nucleotide sequence of the light chain of the anti-PDL1 antibody
atggagacagacacactcctgctatgggtactgctgctctgggttccaggttccactggtgacatccagatgacccagagccctagcagcctgagcgccagcgtgggagatagagtgaccatcacctgcagggcctctcaggacgtgtctacagccgtggcttggtaccagcagaagccaggaaaggcccccaagctgctgatctacagcgcctctttcctgtacagcggcgtgccttctagattcagcggcagcggaagcggcaccgatttcaccctgaccatcagcagcctgcagccagaggacttcgccacctactactgccagcagtacctgtaccacccagccacattcggccagggcaccaaggtggagatcaagaggaccgtggccgcccctagcgtgtttatcttccctcctagcgacgagcagctgaaaagcggaacagccagcgtcgtctgcctgctgaacaacttctaccccagggaggccaaagtccagtggaaagtggacaacgccctgcagagcggcaattctcaggagagcgtgaccgagcaggatagcaaggacagcacctacagcctgagcagcacactgaccctgagcaaggccgactacgagaagcacaaggtgtacgcttgcgaggtcacacaccagggactgagcagccccgtgaccaagagcttcaaccgcggcgagtgttag

SEQ ID NO: 12 - DNA, 861 bp, Artificial Sequence
  The nucleotide sequence of an anti-PDL1 single- chain antibody
atgtaccgcatgcagctgctgtcttgcatcgccctgtctctggctctggtgaccaatagccggatcaccctgaaggagagcggacctcctctggtgaaacctacccagaccctgaccctgacttgcaccttcagcggcttctctatgagcgatccagaggccggagtggcttggatcagacagccaccaggcaaggcactcgagtggctggctatcatctacgccgacggaagcgccgcctattctccttctctgaacacccggctgaccatcaccaaggacaccagcaagaaccaggtggtgctggtcatgaccagagtgtccccagtggataccgccacctacttttgcgcccacagaccaggcagcttcaggctgaacaacgtgtccctggacagcagcgtgtcagcaggatttgccgtgtggggtccaggaatcaccgtgaccatctctagcggaggaggaggaagcggaggaggaggatctggaggaggaggaagcggaggaggaggagcatctgccctgcagctgacacagtctccttctagcctgagcgctagcgtgggcgatagaatcaccatcacttgcgtcgccgacaagaaggtcggctactacatggcttggtaccggcagaagccaggatctcctcctcagctgctggtgtacggaagcgatcagctgtacgagggcgtgcctagcagattcagcggcagcggaagcggaaccgagttcaccctgaccatcagcaccctgagacccgaggacttcgccacctactattgcgcagccctgcacgccctgccttatgtgtttggaccaggcaccagagtggacgtcagacatcatcatcatcatcattaa

SEQ ID NO: 13 - protein, 14 aa, Artificial Sequence
  The amino acid sequence of the signal peptide Sig1 of a secretory anti-PDL1 single-chain antibody
MLLTLIILLPVVSK

SEQ ID NO: 14 - protein, 17 aa, Artificial Sequence
  The amino acid sequence of the signal peptide Sig2 of a secretory anti-PDL1 single-chain antibody
MWLQSLLLLGTVACSIS

SEQ ID NO: 15 - protein, 20 aa, Artificial Sequence
  The amino acid sequence of the signal peptide Sig3 of a secretory anti-PDL1 single-chain antibody
MYRMQLLSCIALSLALVTNS

SEQ ID NO: 16 - protein, 20 aa, Artificial Sequence
  The amino acid sequence of the signal peptide Sig4 of a secretory anti-PDL1 single-chain antibody
METDTLLLWVLLLWVPGSTG

(19) United States
(12) Patent Application Publication
(10) Pub. No.: US 2021/0254102 A1
(43) Pub. Date: Aug. 19, 2021

(54) Preparation Method for Attenuated Rhabdovirus and Application Thereof

(71) Applicant: FANTASIA BIOPHARMA (ZHEJIANG) CO. LTD, Zhejiang (CN)

(72) Inventor: Frank XiaoFeng QIN, Jiangsu (CN)

(73) Assignee: FANTASIA BIOPHARMA (ZHEJIANG) CO. LTD, Zhejiang (CN)

(21) Appl. No.: 17/168,311

(22) Filed: Feb. 5, 2021

Related U.S. Application Data
(63) Continuation of application No. PCT/CN2018/100047, filed on Aug. 10, 2018.

(51) Int. Cl. C12N 15/86; C12N 7/00; C07K 16/28; A61K 45/06; A61K 39/395; A61P 35/04

(52) U.S. Cl. CPC C12N 15/86; C12N 7/00; C07K 16/2827; A61K 45/06; C07K 2317/622; A61P 35/04; C12N 2760/20021; C12N 2760/20043; A61K 39/3955

(57) ABSTRACT

Provided is a preparation method of an attenuated rhabdovirus expression vector, which is used to prepare an attenuated RNA virus recombinant expression vector system for the chimeric expression of an antibody directed against a specific target, wherein the vector system may stably express a corresponding antibody directed against a specific target.

### CROSS-REFERENCE TO EARLIER FILED APPLICATIONS

This application claims the benefit of and is a continuation of PCT/CN2018/100047, filed Aug. 10, 2018, the entire disclosure of which is hereby incorporated by reference.

### INCORPORATION BY REFERENCE

In compliance with 37 CFR 1.52(e)(5), the instant application contains Sequence Listings which have been submitted in electronic format via EFS and which are hereby incorporated by reference. The sequence information contained in electronic file named 6849-180407US-seql.txt, size 73 KB, created on Apr. 21, 2021, using SIPOSequenceListing 1.0, is hereby incorporated by reference in its entirety.

### TECHNICAL FIELD

The present disclosure relates to the fields of oncology, virology, and molecular cell biology. To be specific, the present disclosure relates to a vector system using a non-integrated and replicable negative-strand RNA virus and a construction method thereof, as well as the use of the vector constructed by the above-mentioned method.

### BACKGROUND

In the past decade, the range of choice of therapeutic regimens for cancer patients has changed dramatically. With the understanding of the driver mutations associated with tumor growth and development as well as the research and development of molecular inhibitors that target these specific mutations, a new field of tumor therapy (i.e., precision oncology) has emerged. The theoretical basis of tumor immunotherapy is that the immune system has the ability to recognize tumor-associated antigens and regulate the body to attack tumor cells (highly specific cytolysis). This biological process is very complex and is still under a mass of basic research at present.

In 1990s, many scientific research groups discovered tumor antigens and discovered that T lymphocytes are capable of recognizing these tumor antigens in a major histocompatibility complex-dependent manner. In some cases, antigens usually refer to viral proteins, mutated self-antigens (some of which are oncogenes), derepressed embryonic antigens, and differentiated proteins or autologous normal proteins that are overexpressed. Tumor cells produce and release antigens via a variety of ways, such as intracellular kinases, primary tumor cell necrosis, and the body's response to radiotherapy, chemotherapy or targeting therapy. In addition to antigens, in an environment with the presence of cell stress, hypoxia, depletion of nutrient substances and trauma, dead tumor cells are also able to release a variety of immunogenic molecules, which are capable of binding to cell surface receptors or intracellular receptors (for example, toll-like receptor), thereby triggering an innate immune response. Moreover, specific antigen presenting cells (such as dendritic cells) in tumor microenvironment are capable of phagocytosing dead tumor cells, soluble antigens and CD8⁺ T cells. In order to achieve a better distinguishing effect, a secondary signal recognition system (a signal pathway mediated by costimulatory molecule(s)) is also established in T cell activation. Once activated in the presence of costimulatory molecule(s), T cells are capable of migrating to the tumor microenvironment following the concentration gradient of local chemokine(s). After T cells reach the vicinity of tumor cells, T cell receptors are capable of recognizing the homologous antigen(s) on the surface of the tumor cells via type I MHC-polypeptide complex. T cells are able to release cytotoxic factors (such as granzyme B and perforins), which are capable of regulating the direct lysis of antigen-expressing tumor cells while producing a bystander effect on adjacent non-antigen-expressing tumor cells. In the microenvironment of some tumors, there are a large number of active effector lymphocytes. These tumors generally have relatively good prognosis and show relatively good response to immunotherapy. Despite the presence of a tumor-immune cycle, the tumor that has been formed may escape from the host's immune detection and tumor elimination via a variety of tumor immune mechanisms in hosts.

Moreover, it has been reported in some researches that a large number of immune suppressive cells, such as regulatory CD4⁺-positive T cells, tumor-associated macrophages and myeloid-derived suppressor cells, may accumulate in the tumor microenvironment. These immune cells are able to suppress the activity of active effector T cells. The way in which tumor cells die may determine which kind of immune response will be activated. For example, tumor cell apoptosis may induce T cell tolerance, while tumor cell necrosis or pyroptosis and programmed cell death may induce active tumor-specific T cell response.

Cancer has been the leading cause of death for a long time. The World Health Organization has predicted that malignant tumors will become the “first killer” of human beings in the 21st century long before. Therefore, the control of cancer has become a global focus in health strategies. Although our country is a developing country, the spectrum of disease has changed and China has become the country with the most cancer patients in the world. In recent years, the situation concerning the morbidity and mortality of malignant tumors has become more serious. There are about 1.6 million new cases per year, up to 1.3 million death and more than 2 million current patients. Besides, there will be still an upward trend in the morbidity and mortality of most cancers, which deserves high attention.

Although the cure rate of several malignant tumors has been significantly improved, the results of patients suffering from advanced solid tumors have still remained unchanged cruelly during past decades. Currently, therapies that have been clinically used for solid tumors are utilizing antibodies against tumor immune checkpoints, for example, anti-PD-1/PDL1 antibody therapy and anti-CTLA4 antibody therapy. The key point of these monoclonal antibodies that effectively antagonize immune checkpoint molecules lies in the effective production capacity per unit volume. In addition, the problem of drug resistance encountered by antibodies against immune checkpoints needs to be solved urgently. At present, the research progress of tumor immunotherapy has attracted attention from countries all over the world, and a variety of immune-related tumor therapeutic strategies including T-cell checkpoint inhibitors, oncolytic viruses, chimeric antigen receptor-T cells and the like have been derived. It is well known that a high-efficiency immunotherapy needs to possess the following main features: inducing the generation of a long-lasting clinical response; no typical drug resistance; and inducing the generation of an autoimmune-like toxicity. Clinical oncologists need to acquire an in-depth understanding of the current state of the clinical application of tumor-targeted therapy and tumor immunotherapy, and only in this way can they provide high-quality therapeutic regimens for cancer patients. The theoretical basis of tumor immnuotherapy is that the immune system has the ability to recognize tumor-associated antigens and regulate the body to attack tumor cells (highly specific cytolysis).

As novel tumor therapeutic agents, some recombinant viruses modified by gene editing trigger antitumor immune response via two mechanisms of the viruses, i.e., the killing effect on tumor cells and the induction of systemic antitumor immune response. However, the specific molecular mechanism remains unclear. Some existing research results demonstrate that the mechanism is the combined effects of a variety of factors, such as the induction of cell death by the replication and proliferation of the virus in tumor cells, the interaction with the antiviral elements in tumor cells, and the promotion of intrinsic and spontaneous antitumor immune response or specific antitumor immune response.

Vesicular stomatitis virus (VSV) is a negative-strand RNA virus that infects most mammalian cells and expresses viral protein accounting for up to 60% of the total protein in the infected cells. In nature, VSV infects swines, cattles and horses, and causes chickenpox disease near mouth and feet. Although it has been reported that human may get infected with VSV, VSV has not caused any serious symptoms in humans. VSV encodes five kinds of proteins, including nucleocapsid protein (N), phosphoprotein (P), matrix protein (M), surface glycoprotein (G) and RNA-dependent RNA polymerase (L). Blocking the protein synthesis in the host cell by VSV matrix protein (M) may induce the death of cells.

However, the existing drugs in the prior art still face numerous shortcomings such as long administration period, drug resistance and high price. Accordingly, there is a need to provide a novel drug to overcome the above-mentioned shortcomings.

### SUMMARY

Problems to be solved by the disclosure.

Based on the shortcomings in the prior art, the present disclosure provides an attenuated RNA virus recombinant expression vector system for the chimeric expression of an antibody against a specific target, specifically, an attenuated virus vector system targeting tumor microenvironment (AVTM system). The aforementioned vector system is capable of stably expressing the corresponding antibody against a specific target.

Meanwhile, the present disclosure also relates to the preparation method and application of the attenuated virus vector system mentioned above.

### Means for Solving the Problems

The technical solutions involved in the present disclosure are as follows.

(1) A preparation method of an attenuated virus vector, comprising the following steps:
(S1) mixing a nucleotide sequence of a gene encoding a matrix protein of a vesicular stomatitis virus as set forth in SEQ ID NO:1 (M gene of VSV) with a first vector, and adding a transposase to initiate a transposition reaction;
(S2) mixing a transposition product obtained in step (S1) with a competent bacterium for transformation;
(S3) extracting a plasmid of a bacterium obtained by step (S2) so as to obtain a transpositioned gene encoding the matrix protein of the vesicular stomatitis virus; and
(S4) recombining the gene obtained in step (S3) into a second vector so as to obtain the attenuated virus vector;
wherein a sequence encoding the second vector comprises a genome sequence of the vesicular stomatitis virus;
wherein the first vector is a vector with transposition function;
alternatively, the first vector comprises a sequence as set forth in SEQ ID NO:2;
alternatively, the second vector comprises a sequence as set forth by a sequence numbered EU849003.1 in GENEBANK.
(2) An attenuated virus vector obtained by the preparation method of (1).
(3) The attenuated virus vector according to (2), wherein a sequence encoding the attenuated virus vector comprises a sequence as set forth in SEQ ID NO: 3.
(4) The attenuated virus vector according to (3), wherein a sequence encoding the attenuated virus vector comprises a sequence as set forth in SEQ ID NO: 4.
(5) A cloning backbone vector system, wherein a sequence as set forth in SEQ ID NO:5 is recombined into the vector of (3) in the cloning backbone vector system; wherein a position where the sequence as set forth in SEQ ID NO:5 is inserted into a sequence encoding the vector of (3) is position 4632 in a sequence as set forth in SEQ ID NO: 4.
(6) The cloning backbone vector system according to (5), wherein a sequence encoding the cloning backbone vector system comprises a sequence as set forth in SEQ ID NO:6.
(7) A method for preparing an attenuated monoclonal virus strain, comprising the following steps:
(S1) culturing a first cell to be transfected;
(S2) co-transfecting the cell to be transfected in step (S1) with a plasmid mixture of a plasmid comprising a sequence as set forth in SEQ ID NO:3, a plasmid comprising a sequence as set forth in SEQ ID NO:7 (pN), a plasmid comprising a sequence as set forth in SEQ ID NO:8 (pL) and a plasmid comprising a sequence as set forth in SEQ ID NO:9 (pP);
(S3) extracting a supernatant of a cell mixture obtained after co-transfection in step (S2) and transfecting a second cell to be transfected with the supernatant; and
(S4) culturing and screening the second cell to be transfected that has been transfected in step (S3), so as to obtain the attenuated monoclonal virus strain.
(8) The method according to (7), wherein the plasmid comprising the sequence as set forth in SEQ ID NO:3, the plasmid comprising the sequence as set forth in SEQ ID NO:7 (pN), the plasmid comprising the sequence as set forth in SEQ ID NO:8 (pL) and the plasmid comprising the sequence as set forth in SEQ ID NO:9 (pP) have a weight ratio of 10:4:1:5.
(9) The method according to (7) or (8), wherein the plasmid comprising the sequence as set forth in SEQ ID NO:3 is a plasmid comprising a sequence as set forth in SEQ ID NO:4; alternatively, the second cell to be transfected is a Vero cell; and the first cell to be transfected is a BSR-T7 cell.
(10) The method according to (9), wherein the plasmid comprising the sequence as set forth in SEQ ID NO:4 in step (S2) is a plasmid comprising a sequence as set forth in SEQ ID NO:6.
(11) The method according to (10), wherein an encoding sequence of the plasmid comprising the sequence as set forth in SEQ ID NO:6 in step (S2) further comprises a sequence as set forth in SEQ ID NO:10 and a sequence as set forth in SEQ ID NO:11.
(12) The method according to (10), wherein an encoding sequence of the plasmid comprising the sequence as set forth in SEQ ID NO:6 in step (S2) further comprises a sequence as set forth in SEQ ID NO:12.
(13) The method according to (12), wherein a sequence comprising the sequence as set forth in SEQ ID NO:12 further comprises a signal peptide sequence at 5′-end; the signal peptide sequence is selected from sequences as set forth in SEQ ID NO:13, SEQ ID NO:14, SEQ ID NO:15 and SEQ ID NO:16; preferably, the signal peptide sequence is a sequence as set forth in SEQ ID NO:15.
(14) An attenuated monoclonal virus strain prepared and obtained by the method for preparing an attenuated monoclonal virus strain according to any one of (7) to (13).
(15) A monoclonal antibody secreted by the attenuated monoclonal virus strain according to (14).
(16) A monoclonal antibody comprising fragments encoded by encoding sequences of a sequence as set forth in SEQ ID NO:4, a sequence as set forth in SEQ ID NO:10 and a sequence as set forth in SEQ ID NO:11.
(17) The monoclonal antibody according to (16), wherein the sequence comprising the sequence as set forth in SEQ ID NO:4 is a sequence comprising a sequence as set forth in SEQ ID NO:6.
(18) The monoclonal antibody according to (17), wherein the encoding sequence further comprises a nucleotide sequence encoding a sequence as set forth in SEQ ID NO:13, SEQ ID NO:14, SEQ ID NO:15 or SEQ ID NO:16; preferably, the encoding sequence further comprises a nucleotide sequence encoding the sequence as set forth in SEQ ID NO:15.
(19) A pharmaceutical composition comprising the monoclonal antibody of any one of (15) to (18).
(20) Use of the monoclonal antibody of any one of (15) to (18) or the pharmaceutical composition of (19) in preparation of a drug for killing abnormally proliferating cells, inducing and promoting antitumor immune response or eliminating immunosuppression in a microenvironment of a tumor tissue.
(21) The use according to (20), wherein the abnormally proliferating cells are contained in the body of a patient.
(22) The use according to (21), wherein the abnormally proliferating cells are selected from tumor cells or tumor tissue-related cells; preferably, the tumor cells are cancer cells; and more preferably, the cancer cells are metastatic cancer cells.
(23) A method for slowly and continuously killing abnormally proliferating cells, comprising a step of contacting the abnormally proliferating cells with the monoclonal antibody of any one of (15) to (18) or the pharmaceutical composition of (19).
(24) The method according to (23), wherein the abnormally proliferating cells are contained in the body of a patient.
(25) The method according to (23), wherein the abnormally proliferating cells are selected from tumor cells or tumor tissue-related cells; preferably, the tumor cells are cancer cells; and more preferably, the cancer cells are metastatic cancer cells.
(26) The method according to (23), wherein the monoclonal antibody of any one of (15) to (18) or the pharmaceutical composition of (19) is administered to a patient.
(27) The method according to (23), wherein the monoclonal antibody of any one of (15) to (18) or the pharmaceutical composition of (19) is administered via one or more administration modes selected from the group consisting of intraperitoneal administration, intravenous administration, intraarterial administration, intramuscular administration, intradermal administration, intratumoral administration, subcutaneous administration and intranasal administration; preferably, administration routes of the administration modes include one or more selected from the group consisting of endoscopy, celioscopy, intervention, minimal invasive surgery and traditional surgery.
(28) The method according to (23), wherein the method further comprises a step of administering a second antitumor therapy.
(29) The method according to (28), wherein the second antitumor therapy is one or more selected from the group consisting of chemotherapy, radiotherapy, immunotherapy and surgical therapy.
(30) A polynucleotide comprising a sequence as set forth in SEQ ID NO:6.

### Advantageous Effects of the Disclosure

The host cells of the AVTM vector system of the present disclosure derive from a variety of sources. Due to the presence of surface glycoprotein (G), this virus system is capable of entering host cells without the mediation by a specific receptor, infecting almost all mammalian cells, and accomplishing the replication of the virus and realizing the high-efficiency expression of an exogenous chimeric gene, thereby significantly enhancing the in-vivo and in-vitro expression efficiency of exogenous chimeric antibody-like substances.

The genome of the rhabdovirus corresponding to the attenuated virus vector system AVTM involved in the present disclosure is a single-stranded negative-strand RNA. Meanwhile, the expression of exogenous genes by this system is very stable. This attenuated virus vector system would not integrate into the genome of a cell, the genome of this virus vector system itself is simple and stable, and the mutation rate of the genome of this virus vector system is low.

The system involved in the present disclosure enables rapid and efficient chimeric expression of human-derived specific antibodies, and enables the expression and external secretion of specific antibodies against tumor cells while completing specific replication in tumor cells. At the same time, while tumor antigens are released to activate the specific antitumor immune response of immune cells, a large number of specific de-immunosuppressive antibodies accumulate in local tumor microenvironment in a short period of time, thereby effectively breaking the local immune suppression barrier, activating the specific killing activity of T cells, promoting the elimination of tumor cells by killer T cells, and promoting the generation of systemic and specific antitumor memory immune response in the body in the meantime.

Since the gene of the AVTM of the present disclosure has been genetically modified to have a genetic mutation, the toxicity of the virus is thus reduced and expressing an antibody at 37° C. would not cause obvious damage to the normal cells of the host. Therefore, infected cells are capable of continuously expressing secretory antibodies within a period of time. On the contrary, as for myeloma cells transfected traditionally, a cell strain used for expressing a specific antibody needs to be replicated to a high order of magnitude before being used for scale production of the antibody.

In one embodiment of the present disclosure, the nucleotide sequence encoding an antibody against an immune checkpoint molecule is inserted into a modified virus expression vector by means of gene editing and recombined in a specific eukaryotic cell, thereby obtaining an attenuated virus system that stably expresses a chimeric antibody.

In one embodiment of the present disclosure, an AVTM-scFV vector system capable of efficiently expressing single-chain antibodies in tumor tissues is obtained by the optimization and screening of the signal peptide sequences for antibody secretion. Meanwhile, this system is further used in a solid tumor model to evaluate the therapeutic efficacy of this recombined system, which provides novel technical solutions and options for the development of therapeutic products for solid tumors.

### BRIEF DESCRIPTION OF THE DRAWINGS

FIG. 1A shows a screening library of attenuated virus strains established by random base insertion of exogenous bases in a rhabdovirus-based vector system and illustrates a schematic diagram of specific process of screening a quadruple-mutant attenuated strain RV-Mut4.

FIG. 1B shows a schematic diagram of a vector obtained by modifying the core backbone of the rhabdovirus-based vector pRV-2MCS, and the modified system is capable of simultaneously integrating two different exogenous genes (i.e., the gene of the heavy chain of an anti-PDL1 antibody and the gene of the light chain of the anti-PDL1 antibody).

FIG. 2 shows schematic diagrams illustrating the simultaneous expression of two exogenous genes by the RV-2MCS vector system. Among them, part A of FIG. 2 shows a schematic diagram in which the heavy chain and the light chain of an anti-PDL1 antibody are expressed respectively; part B of FIG. 2 shows a schematic diagram in which the heavy chain and the light chain of an anti-PDL1 antibody are simultaneously expressed by the RV-2MCS vector system; and part C of FIG. 2 shows a schematic diagram of the expression of fluorescent proteins by the RV-2MCS vector system (simultaneously integrating green fluorescent protein (GFP) and red fluorescent protein (RFP)) in Vero cells.

FIG. 3 shows schematic diagrams of the simulation, analysis and optimization of the codon preference of the AVTM vector system for chimeric expression of an anti-PDL1 single-chain antibody. Among them, part A of FIG. 3 shows the average GC content in a designed and optimized exogenous gene sequence (58%); part B of FIG. 3 shows the codon preference in mammalian cells, and the corresponding codon adaption index (CAI) is 0.95; and part C of FIG. 3 shows a schematic diagram illustrating the optimal distribution pattern of a variety of codons in mammalian cells and the relative distribution frequency of the synonymous codons of amino acids in the gene sequence.

FIG. 4 shows the superiority of the RV-G21E-M51A-L111F-V221F (RV-Mut4) quadruple-mutant vector system compared to other systems. Among them, part A of FIG. 4 and part B of FIG. 4 show schematic diagrams demonstrating that the RV-Mut4 vector has the characteristic of continuously expressing an exogenous protein for a long period of time in cells in vitro; part C of FIG. 4 shows a schematic diagram of curves which are plotted based on FACS-based detection method and illustrate the time-dependent change of the amount of exogenous GFP protein expressed by RV-Mut4 and other mutant strains upon their replication in Vero cells; and part D of FIG. 4 shows a schematic diagram illustrating the results of the comparative experiments on the toxicity of RV-Mut4 and other three mutant strains to tumor cells and said toxicity is determined by MTT assay.

FIG. 5 shows a schematic diagram illustrating the binding ability of an anti-PDL1 single-chain antibody to the surface molecules of two kinds of tumor cells (LLC cells and MC38 cells) determined by FACS, wherein the single-chain antibody is an exogenous single-chain antibody externally secreted by an engineered Vero cell line (RV-Mut4-scFV-PDL1 that highly expresses the anti-PDL1 single-chain antibody) and the antibody in the supernatant is co-incubated with two kinds of cells that stably express human PDL1 (LLC cells and MC38 cells) in vitro.

FIG. 6 shows a schematic diagram illustrating the external secretion level of the single-chain antibodies against the immune checkpoint molecule PDL1 in in-vitro cell lines determined by IB (immunoblotting) assay in eukaryotic cells, wherein the single-chain antibodies are linked to and mediated by three different kinds of signal peptides.

FIG. 7A shows a schematic diagram illustrating the secretion of the anti-PDL1 single-chain antibodies into the supernatant, wherein said anti-PDL1 single-chain antibodies are derived from four single-chain antibodies mediated by the AVTM vector system in engineered Vero cells.

FIG. 7B shows a schematic diagram illustrating the detection of the presence of a single-chain antibody linked to a signal peptide (signal 3) in serum and local tumor tissue after LLC animal models are inoculated with an attenuated strain expressing said antibody in two different inoculation approaches (subcutaneous injection adjacent to the tumor tissue and intratumoral injection).

FIG. 8 shows schematic diagrams illustrating the evaluation of the therapeutic effect of an AVTM-mediated single-chain antibody in lung cancer. As shown in part A of FIG. 8, there are three treatment groups, namely, AVTM system-mediated single-chain antibody group (RV-scFV-PDL1 attenuated strain), RV-WT experimental control group and PBS blank control group. Part A of FIG. 8 illustrates the overall statistical diagram showing the therapeutic effect on solid tumor in LLC non-small cell lung cancer model (mouse model). In order to further determine the inter-individual difference among different treatment groups, individuals in each group mentioned above are subjected to statistical analysis separately. Part B of FIG. 8 shows an individual statistical diagram of PBS treatment group. Part C of FIG. 8 shows a schematic individual statistical diagram of RV-GFP-WT treatment group. Part D of FIG. 8 shows an individual statistical diagram of RV-scFV-PDL1 treatment group. Model mice were inoculated intratumorally (i.e., in the tumor tissues of non-small cell lung cancer models) with 10⁷ PFU (30 μl) of the attenuated virus every other day (three times in total). Tumor volume changes of the model mice in each experimental group were measured every other day. Parts B to D of FIG. 8 show the changes of the tumor volumes of the individuals in three groups over time, respectively.

Parts A to C of FIG. 9 are schematic diagrams illustrating the therapeutic effects on individuals exerted by PBS, RV-GFP and RV-scFV-PDL1 in lung cancer model, respectively. Part D of FIG. 9 is a statistical diagram of the therapeutic effects of the above three treatment groups on Day 10. Part E of FIG. 9 is a statistical diagram of the therapeutic effects of the above three treatment groups on Day 20.

FIG. 10 shows the evaluation of the effect of the attenuated strain RV-scFV-PDL1 in metastatic lung cancer animal model, that is, the metastasis observed in LLC-JSP mouse models that show metastasis in lung tissues and are treated in the experimental groups and the control groups under a low-magnification microscope (part A of FIG. 10) and the survival rate of the model mice (part B of FIG. 10).

FIG. 11 shows a schematic diagram illustrating the tumor volume changes recorded in a case where a human CD274 colon cancer mouse model (expressing human PDL1) is first established, RV-scFV-PDL1 and the corresponding control virus (RV-WT) are intratumorally administered at a dose of 30 μl (10⁷ PFU) every other day (three times in total).

### DETAILED DESCRIPTION

### Definitions

In the claims and/or specification of the present disclosure, the wording “a”, “an” or “the” may refer to “one”, but may also refer to “one or more”, “at least one” and “one or more than one”.

As used in claims and specification, the wording “comprise”, “have”, “include” or “contain” means inclusive or open-ended, and does not exclude additional and unreferenced elements, method or steps. Meanwhile, the wording “comprise”, “have”, “include” or “contain” may also mean close-ended, excluding additional and unreferenced elements, method or steps.

Throughout the application document, the term “about” means that a value includes the standard deviation of the error of the device or method used to determine the value.

Although the definition of the term “or” as being an alternative only and as “and/or” are both supported by the disclosed content, the term “or” in claims means “and/or” unless it is explicitly indicated that the term “or” only means an alternative or the alternatives are mutually exclusive.

When used in claims and/or specification, the term “inhibition”, “reduction”, “prevention” or any variation of these terms includes any measurable reduction or complete inhibition for the purpose of achieving the desired results (for example, treatment of tumor). Desired results include but are not limited to the relief, reduction, slowing or eradication of a cancer, a hyperproliferative condition or a symptom related to a cancer, as well as the improved quality or extension of life.

The vaccination method of the present disclosure may be used for treating tumors in a mammal. Alternatively, the vaccination method of the present disclosure may be used for treating cancers in a mammal. The term “cancer” used in the present disclosure includes any cancer, including but not limited to melanoma, sarcoma, lymphoma, cancer (for example, brain cancer, breast cancer, liver cancer, gastric cancer, lung cancer, and colon cancer) and leukemia.

The term “mammal” refers to human and non-human mammals.

The method of the present disclosure comprises administering to a mammal an oncolytic vector expressing a tumor antigen to which the mammal has pre-existing immunity. The term “pre-existing immunity” used in the present disclosure is intended to include the immunity induced by vaccination with an antigen and the immunity naturally existing in a mammal.

The term “RV virus” used in the present disclosure refers to an attenuated VSV oncolytic rhabdovirus. The term “RV-Mut” refers to an oncolytic rhabdovirus having mutation(s) compared to the wild-type VSV oncolytic rhabdovirus. The term “RV-Mut4” refers to an oncolytic rhabdovirus with mutations at four sites as compared with the wild-type VSV oncolytic rhabdovirus.

The term “VSV” refers to vesicular stomatitis virus, which is one kind of oncolytic rhabdoviruses and encodes five kinds of proteins, including nucleocapsid protein (N), phosphoprotein (P), matrix protein (M), surface glycoprotein (G) and RNA-dependent RNA polymerase (L).

The term “PDL1” refers to cell programmed death ligand 1. PDL1 protein is the ligand of PD1, and is associated with the suppression of the immune system and capable of transmitting inhibitory signals. Once PD1 binds to PDL1, a negative regulatory signal will be transmitted to T cells, induce T cells into a resting state and reduce the proliferation of CD8⁺ T cells in lymph nodes, thus making T cells unable to recognize cancer cells and reducing the proliferation of T cells or resulting in the apoptosis of T cells.

The term “vaccine” in the present disclosure refers to an immune formulation for preventing diseases prepared by methods such as artificially attenuating, inactivating or genetically modifying pathogenic microorganisms (such as bacteria) and the metabolites thereof.

The term “radiotherapeutic agent” in the present disclosure includes drugs that cause DNA damage. Radiotherapy has been widely used in the treatment of cancer and diseases, and includes those commonly referred to as γ-ray and X-ray and/or targeted delivery of radioisotopes to tumor cells.

The term “chemotherapeutic agent” in the present disclosure is a chemical compound useful for treating cancer. Classes of chemotherapeutic agents include but are not limited to: an alkylating agent, an antimetabolite, a kinase inhibitor, a spindle poison plant alkaloid, a cytotoxic/antitumor antibiotic, a topoisomerase inhibitor, a photosensitizer, an anti-estrogen, a selective estrogen receptor modulator, an anti-progesterone, an estrogen receptor downregulator, an estrogen receptor antagonist, a luteinizing hormone-releasing hormone agonist, anti-androgens, an aromatase inhibitor, an EGFR inhibitor, a VEGF inhibitor, an antisense oligonucleotide that inhibits the expression of gene(s) involved in abnormal cell proliferation or tumor growth. Chemotherapeutic agents that may be used in the treatment method of the present disclosure include a cell growth inhibitor and/or a cytotoxic agent.

The term “immunotherapeutic agent” in the present disclosure comprises an “immunomodulator” and an agent that facilitates or mediates an antigen presentation that increases a cell-mediated immune response. Among them, the “immunomodulator” comprises an immune checkpoint modulator. For example, immune checkpoint protein receptors and their ligands mediate the suppression of T cell-mediated cytotoxicity and are often expressed by tumors or expressed on anergic T cells in the tumor microenvironment, thus permitting the tumor to evade immune attack. Inhibitors of the activity of immunosuppressive checkpoint protein receptors and their ligands may overcome the immunosuppressive tumor environment, so as to permit cytotoxic T cell attack on tumor. Examples of immune checkpoint proteins include but are not limited to PD-1, PD-L1, PDL2, CTLA4, LAG3, TIM3, TIGIT and CD103. Modulation (including inhibition) of the activity of such protein may be accomplished by an immune checkpoint modulator, which may include, for example, an antibody, an aptamer, a small molecule, a soluble form of a checkpoint receptor protein and the like that target a checkpoint protein. PD-1-targeting inhibitors include the approved drug agents pembrolizumab and nivolumab, while ipilimumab is an approved CTLA-4 inhibitor. Antibodies specific for PD-L1, PD-L2, LAG3, TIM3, TIGIT and CD103 are known and/or commercially available, and may also be produced by those skilled in the art.

As for the “conventional biological methods in this field” in the present disclosure, please refer to the corresponding methods described in the public publications such as “Current Protocols in Molecular Biology” published by Wiley, “Molecular Cloning: A Laboratory Manual” published by Cold Spring Harbor Laboratory.

The specific meanings of the nucleotide/amino acid sequences involved in the present disclosure are as follows.

The sequence as set forth in SEQ ID NO:1 is the nucleotide sequence of the M gene in VSV core backbone (i.e., the M gene in pRV-Core vector).

The sequence as set forth in SEQ ID NO:2 is the nucleotide sequence of Entranceposon, a vector with the function of transposition.

The sequence as set forth in SEQ ID NO:3 is the nucleotide sequence of the M gene in the attenuated virus vector obtained by the method of the present disclosure.

The sequence as set forth in SEQ ID NO:4 is the nucleotide sequence of pRV-core Mut4 vector.

The sequence as set forth in SEQ ID NO:5 is the nucleotide sequence of 2MCS.

The sequence as set forth in SEQ ID NO:6 is the nucleotide sequence of pRV-2MCS vector.

The sequence as set forth in SEQ ID NO:7 is the nucleotide sequence of a plasmid comprising the N gene in VSV core backbone.

The sequence as set forth in SEQ ID NO:8 is the nucleotide sequence of a plasmid comprising the L gene in VSV core backbone.

The sequence as set forth in SEQ ID NO:9 is the nucleotide sequence of a plasmid comprising the P gene in VSV core backbone.

The sequence as set forth in SEQ ID NO:10 is the nucleotide sequence of the heavy chain of the anti-PDL1 antibody.

The sequence as set forth in SEQ ID NO:11 is the nucleotide sequence of the light chain of the anti-PDL1 antibody.

The sequence as set forth in SEQ ID NO:12 is the nucleotide sequence of an anti-PDL1 single-chain antibody.

The sequence as set forth in SEQ ID NO:13 is the amino acid sequence of the signal peptide Sig1 of a secretory anti-PDL1 single-chain antibody.

The sequence as set forth in SEQ ID NO:14 is an amino acid sequence of the signal peptide Sig2 of a secretory anti-PDL1 single-chain antibody.

The sequence as set forth in SEQ ID NO:15 is an amino acid sequence of the signal peptide Sig3 of a secretory anti-PDL1 single-chain antibody.

The sequence as set forth in SEQ ID NO:16 is an amino acid sequence of the signal peptide Sig4 of a secretory anti-PDL1 single-chain antibody.

### EXAMPLES

Other objects, features and advantages of the present disclosure will become apparent from the following detailed description. However, it should be understood that the detailed description and specific Examples (although representing the specific embodiments of the present disclosure) are given for explanatory purposes only, since various changes and modifications made within the spirit and scope of the present disclosure will become apparent to those skilled in the art after reading this detailed description.

The sources of reagents and consumables used in the present disclosure are as follows.

PBS (Hyclone SH30256.01), DMEM high glucose medium (Gibco C11995500), RPM11640 (Gibco C22400500CP), double antibody (Gibco 15140-122), fetal bovine serum (Gibco 10099141), Opti-MEM® I Reduced Serum Medium (Gibco 31985-070), Lipofectamine LTX (Invitrogen 15338100), 96-well cell culture plate (Corning 3599), 6-well cell culture plate (Corning 3516), 0.22 μm filter (Millipore SLGP033rb), DMSO (Macklin D806645), and thiazolyl blue (Sigma M2128).

Unless otherwise specified, all the reagents mentioned in the Examples of the present disclosure are commercially available.

The method for virus packaging involved in the present disclosure was as follows.

1. BSR-T7 cells were seeded in a 6-well plate to enable the cell density to reach 3×10⁵ cells/well. A poxvirus expressing T7 polymerase was added 14 h to 16 h after the seeding, and transfection was carried out after the cells were infected with the virus for 6 h.
2. The plasmids were diluted with 200 μl of opti-MEM culture medium, in which the total weight of the plasmids was 5 μg, and the mass ratio of the plasmids was as follows: pRV-core:pP:pN:pL=10:5:4:1. 7.5 μl of the transfection reagent PLUS Reagent (Life Corporation) was further added. 10 μl of Lipofectamine LTX was diluted with 200 μl of the culture medium. pN represented a plasmid expressing the nucleoprotein of the rhabdovirus, pL represented a plasmid expressing the polymerase protein of the rhabdovirus, pP represented a plasmid expressing the phosphoprotein of the rhabdovirus, and the parent vector corresponding to the three plasmids (pP, pL and pN) was pCAGGS (purchased from ATCC).
3. 200 μl of LTX mixed liquid and 200 μl of DNA mixed liquid were mixed and incubated at room temperature for 15 min.
4. The culture medium in the 6-well plate was replaced with Opti-MEM culture medium. The resulting mixed liquid obtained in step 3 was added dropwise into the 6-well plate in which cells were cultured, and the 6-well plate was shaken gently so as to allow the mixed liquid to be evenly distributed in the 6-well plate.
5. After 6 h to 8 h of transfection, the transfection reagent was aspirated and removed, and 3 ml of fresh complete culture medium was added.
6. The cell supernatant was harvested after 72 h and filtered with a 0.22-μm filter.

### Example 1: Establishment of a Screening Library of Attenuated Virus Vectors

In this Example, a kit produced by Thermo Co. (i.e., phage Mu transposition technique) was used, and this technique had been widely used in the research of the functions of various viral genomes and the interaction between viruses and hosts. The principle of establishing a random mutation library by using the phage Mu transposition kit was as follows. Phage Mu transposon-mediated high-density random insertion mutagenesis technique was a method of randomly inserting a 15 bp short nucleotide sequence (5′-NNNNNTGCGGCCGCA-3′, wherein the five N represented five repeated base sequences in the target DNA sequence) in DNA so as to generate a gene insertion mutant library with high storage capacity. The corresponding virus mutant library could be obtained by the combination of such technique and genetic manipulation techniques related to virus. Further combination of techniques such as PCR amplification, capillary electrophoresis, fluorescence-labeled DNA sequencing technique and fragment length polymorphism analysis (AFLP) enabled accurate identification of the number of mutations and the insertion sites. The plasmid DNA involved in the transformation was M1-M mut. That is, the mutation sequences in the mutation sequence library of the M gene were integrated into the M1-kan vector, thereby forming a mutation library of M gene (please refer to the standard instruction of Thermo Scientific Mutation Generation System Kit for the experimental steps). With the combined action of the transposon and the transposase, fragments would be randomly inserted among the bases of M gene in the vector plasmid carrying the target M gene, and finally, random mutations of orders of magnitude were generated in M gene. 5′-TGCGGCCGCA-3′ (a specific primer) was used to amplify the mutated M gene, a mutation library was established, and the genes in the M gene library were further digested and cloned into the pRV-core core backbone plasmid.

The experimental method and steps for establishing a virus library were as follows.

1. The reaction was carried out in a centrifuge tube, wherein the transposase should be added finally. The in-vitro transposition reaction system was as shown in Table 1.

TABLE 1
In-vitro transposition reaction system

| Reagents | Volume |
|---|---|
| Target DNA (M gene in the core backbone of | 1 μl to 14 μl |
| rhabdovirus) | |
| 5X transposase reaction buffer | 4 μl |
| 100 ng of M1-Kan transposon (Entranceposon) | 1 μl |
| MuA transposase | 1 μl |
| H₂O | added to a total |
| | volume of 20 μl |

The positive control DNA (that is, 370 ng (1 μl) of pBlank) provided in Thermo Scientific Mutation Generation System Kit (purchased from Thermo Co., Catalog No. F701) was used for a positive control reaction.

2. The reactants were mixed gently.
3. The resultant was incubated at 30° C. for 1 hour for transposition.
4. The resultant was incubated at 75° C. for 10 minutes to inactivate the transposase.
5. A competent bacterium was prepared, wherein the bacterium was derived from DH5a. The reaction mixture obtained in step 4 was diluted 10 times in deionized water and the maximum volume used each time was 10 μl. Or, the DNA in the reaction mixture was first precipitated and then suspended in deionized water and used for electroporation. As for chemical transformation, 5 μL to 10 μL of said reaction mixture was used each time.
6. 10 μg/ml chloramphenicol or 10 μg/ml kanamycin was added on an agar plate, and an antibiotic against the target DNA clone was further added.
7. Plasmids were extracted from the colonies on the plate by using a standard alkaline lysis method or any commercial DNA preparation kit.
8. The plasmid DNA was digested with restriction enzymes. The monoclonal restriction site (NotI) of the shuttle fragment (provided by Thermo Scientific Mutation Generation System Kit) was determined and the plasmid was digested with two kinds of restriction enzymes to obtain a part of the vector backbone, so as to distinguish these fragments via agarose gel electrophoresis.
9. The presence of the shuttle vector was detected by standard agarose gel electrophoresis, and the pTrans plasmid of the aforementioned shuttle vector was obtained by gel recovery.
10. The extracted DNA fragments were cloned into a new cloning vector M1-CAM^{R} (provided by the kit).
11. Transformation was carried out on an agar plate which was supplemented with 10 μg/ml chloramphenicol or 10 μg/ml kanamycin and further added with an antibiotic against the cloning vector.
12. Colonies were picked from the plate and subjected to amplification. The plasmid DNA was extracted by using a standard alkaline lysis method or any commercial DNA preparation kit.
13. The plasmids were digested with NotI to remove the shuttle vector. Standard agarose electrophoresis could be used to separate the resulting fragments. Fragments with the size of the target DNA were extracted from the cloning vector.
14. The enzyme-digested products obtained by the digestion of NotI were used for ligation. If the enzyme-digested products were not subjected to separation by agarose electrophoresis, the concentration of the DNA mixture was adjusted to 1 ng/ml to 5 ng/ml in order to facilitate the self-ligation of the enzyme-digested products.
15. Escherichia coli DH5α competent cells were used for transformation.
16. The plate was merely added with an antibiotic for screening the cloning vector. Neither chloramphenicol nor kanamycin was added after the shuttle vector was removed via the digestion by NotI.

17. Colonies were picked from the plate, the bacteria were cultured, the plasmid DNA was extracted, and M mut gene fragment was further obtained from the M1-M mut plasmid by enzyme digestion. The establishment of the mutation library of M gene was completed in accordance with the above steps.

The steps of the virus rescue of the attenuated strain were as follows. After the establishment of the mutation library, the mutant fragment M1-M mut was further cloned into the backbone vector pRV-core, wherein pRV-core was the core backbone vector for virus packaging, the whole backbone of the virus was obtained by gene synthesis and the sequence was as set forth by the sequence of pRV-4Mut listed in the present disclosure, and the virus gene corresponding to the pRV-core core backbone was synthesized with reference to the gene of the MuddSummer strain of VSV (GenBank No.: EU849003.1). As compared with the pRV-core plasmid, pRV-4Mut only had non-synonymous mutations at four amino acid sites in M gene, and pRV-core was the original backbone vector. The wild-type M gene in the target vector had been removed via double enzyme digestion, a core backbone vector library comprising the new mutation library of M gene was formed, and this mutant plasmid library was named pRV-core Mut. Further, with the aid of the helper plasmid, a variety of recombinant RV-Mut attenuated strains were obtained by virus rescue in BSR-T7 cells.

The specific steps of the aforementioned virus rescue were as follows. A calcium phosphate transfection kit was used, and BSR-T7 cells (purchased from ATCC) were transfected with 5 μg of the backbone plasmid pRV-core Mut by cell transfection technique. The aforementioned plasmids were diluted with 200 μl of opti-MEM culture medium, the total weight of the plasmids was 5 μg, the ratio of the plasmids was as follows: pRV-core:pP:pN:pL=10:5:4:1. 7.5 μl of the transfection reagent PLUS Reagent (Life Corporation) was further added. 10 μl of Lipofectamine LTX was diluted with 200 μl of the culture medium. Among them, pN represented a plasmid expressing the nucleoprotein of the rhabdovirus, pL represented a plasmid expressing the polymerase protein of the rhabdovirus, pP represented a plasmid expressing the phosphoprotein of the rhabdovirus, wherein the parent vector corresponding to the three plasmids (pP, pL and pN) was pCAGGS (purchased from ATCC). Cells were washed twice by PBS after 6 hours and further cultured in DMEM supplemented with 10% fetal bovine serum for 3 days. The resulting cell supernatant were co-incubated with Vero cells at 37° C. for 3 days, and the virus rescue was confirmed by observing the green fluorescence in the cells under a fluorescence microscope. Further, the rhabdoviruses in the rescued mutant rhabdovirus library were passaged in Vero cells, and monoclonal virus strains were selected in the established plaque screening system.

During the reaction of this transposition system, the in-vitro transposon was not affected by exogenous DNA and the accompanying minor impurities. The optimal amount of the target DNA for each reaction depended on the size of the plasmid. The amount of the target DNA could be calculated by the following formula: the amount of the target DNA (ng)=the size of the plasmid used for reaction (kb)×40 ng.

DNA and the shuttle vector could be inserted into the cloning vector together via DNA cloning technique. DNA was cut from the cloning vector by the restriction enzyme, and there was no such restriction site on the shuttle vector. In addition, the target fragment could be separated by agarose gel electrophoresis due to the distinct size difference between the cloning vector and the inserted DNA.

In order to obtain clones with the maximum amount of insertion mutation, transformation efficiency was also a very important consideration (for example, for pUC19 plasmid, electroporated cells>10⁸ cells/μg). Therefore, electroporation method was the best transformation method selected by the present inventors. Meanwhile, in view of the fact that the shuttle vector comprised inverted terminal repeats (50 bp), RecA produced by Escherichia coli was used as the homologous recombinase so as to avoid potential homologous recombination between these repeats.

After the pRV-core Mut plasmid was obtained, the specific steps of virus rescue were as follows. 5×10⁶ BSR-T7 cells were seeded evenly in a 10-cm cell culture dish and cultured overnight in DMEM supplemented with 10% fetal bovine serum until the cell density reached 80%. Cells were washed with serum-free DMEM twice one hour before transfection. Using a calcium phosphate transfection kit, cells were transfected with 5 μg of the backbone plasmid pRV-core Mut, 5 μg of pN (a plasmid expressing the nucleoprotein of the rhabdovirus), 2.5 μg of pL (a plasmid expressing the polymerase protein of the rhabdovirus), 2.5 μg of pP (a plasmid expressing the phosphoprotein of the rhabdovirus), and the parent vector corresponding to the three plasmids (pP, pL and pN) was pCAGGS (purchased from ATCC). After 2 h to 3 h, cells were washed twice with PBS and cultured in DMEM supplemented with 10% fetal bovine serum for 3 days. The resulting cell supernatant were co-incubated with Vero cells at 37° C. for 3 days, and the virus rescue was confirmed by observing the green fluorescence in the cells under a fluorescence microscope. Further, the rhabdoviruses in the rescued mutant rhabdovirus library were passaged in Vero cells, and monoclonal virus strains were selected in the established plaque screening system. Furthermore, the selected monoclonal virus strains were used to infect new Vero cells, and monoclonal virus strains with reduced ability to lyse cells were selected.

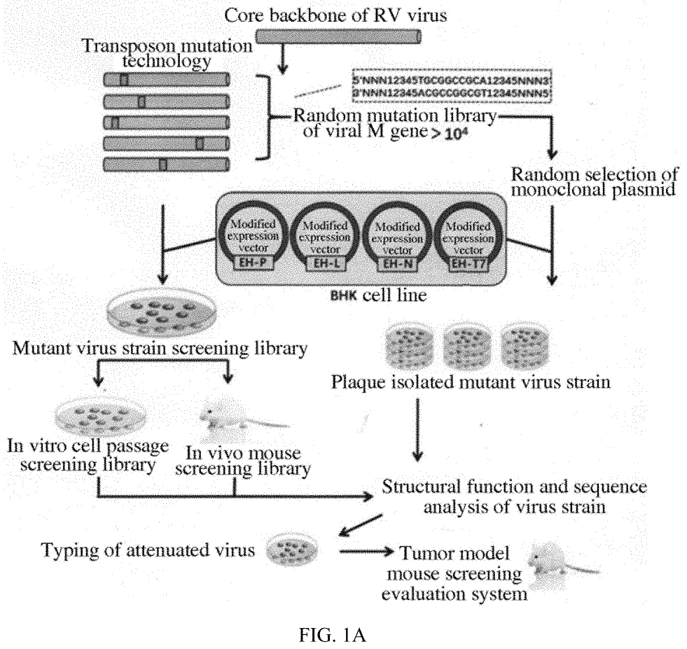

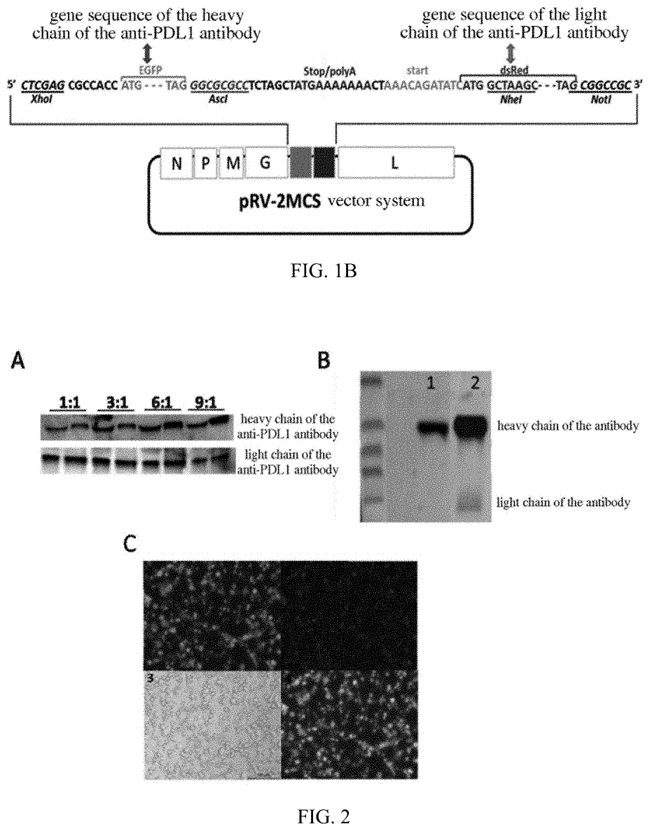

The specific operating steps of the cloning of a backbone plasmid system were as follows. The gene fragment 2MCS (SEQ ID NO:5) was cloned to the vector pRV-core Mut4 by gene synthesis technique, and the corresponding core backbone was pRV-core (pRV-core Mut4 had mutations at four sites on the M gene corresponding to the pRV-core plasmid. Specifically, in M protein, amino acid G at position 21 was mutated to amino acid E, amino acid M at position 51 was mutated to amino acid A, leucine L at position 111 was mutated to phenylalanine F, and valine V at position 221 was mutated to phenylalanine F), and the new backbone obtained from cloning was named pRV-2MCS. The upstream restriction site of the clone was XhoI and the downstream restriction site was NotI (see FIG. 1B), and 2MCS gene was located between G gene and L gene in the vector.

Experimental results: FIG. 1A illustrated the scheme of establishing a random mutation library of the M gene of the rhabdovirus after the random insertion of exogenous bases by the method for establishing the above-mentioned virus library. Further, as shown in FIG. 1A, a screening library of attenuated virus strains was established by using the rhabdovirus-based vector system (see steps 1 to 17 in Example 1). The attenuated strains with reduced replication ability obtained by the above-mentioned method included RV-M51R (a single-mutant strain), RV-M51R-V221F (a double-mutant strain), RV-G21E-M51R-L111F (a triple-mutant strain), RV-G21E-M51A-L111F (a triple-mutant strain) and a quadruple-mutant strain RV-Mut4 (RV-G21E-M51A-L111F-V221F). The virulence genes of RV-Mut4 were four mutations in the M gene of VSV virus corresponding to four amino acid mutations in M protein (the corresponding virus core backbone plasmid was pRV-core Mut4), that is, in M protein, amino acid G at position 21 was mutated to amino acid E, amino acid M at position 51 was mutated to amino acid A, leucine L at position 111 was mutated to phenylalanine F, and valine V at position 221 was mutated to phenylalanine F.

FIG. 1B illustrated a schematic diagram of the RV-Mut4 virus-based core backbone vector pRV-core Mut4. Further, 2MCS gene as shown in FIG. 1B was recombined into the specific site of the pRV-core Mut4 core backbone plasmid (that is, the spacer sequence between G gene and L gene of the virus) by gene synthesis and molecular cloning technique (meanwhile, spacer sequences between the two exogenous genes were cloned, and each spacer sequence had single and specific restriction site), and the new backbone plasmid system was named pRV-2MCS.

The specific steps of cloning the pRV-2MCS core backbone plasmid were as follows. The gene fragment 2MCS was digested and cloned into the plasmid pRV-core Mut4 (in the spacer sequence between G gene and L gene of the virus) by gene synthesis technique, and the new core backbone plasmid obtained after the recombination was pRV-2MCS (FIG. 1B).

### Example 2: Simultaneous Expression of Specific Antibodies by Utilizing the Attenuated Virus Vector System pRV-2MCS

Experimental methods: Based on the plasmid system pRV-2MCS prepared in Example 1, the sequence of the heavy chain and the sequence of the light chain of an anti-PDL1 antibody were respectively cloned into the backbone plasmid pRV-2MCS to form new cloning vectors pRV-PDL1-H and pRV-PDL1-L. The recombinant viruses were rescued, and the corresponding intact and active anti-PDL1 antibodies were expressed and secreted in engineered Vero cells at different ratios of the heavy chain of the anti-PDL1 antibody to the light chain of the anti-PDL1 antibody.

The specific steps of the above-mentioned method were as follows. The complete sequence of the anti-PDL1 antibody was consistent with the anti-PDL1 antibody from Roche Co. The heavy chain gene of the anti-PDL1 antibody (PDL1-H) (gene synthesis was completed by Synbio Technologies) was cloned into the backbone plasmid pRV-2MCS at specific restriction sites (NheI and NotI) to form a new core backbone plasmid pRV-PDL1-H. Similarly, the synthesized sequence of the light chain of the anti-PDL1 antibody was cloned into the plasmid pRV-2MCS by double restriction digestion at specific restriction sites (XhoI and AscI) to form a new core backbone plasmid pRV-PDL1-L. The recombinant viruses RV-PDL1-H (capable of the chimeric expression of the heavy chain of the anti-PDL1 antibody) and RV-PDL1-L (capable of the chimeric expression of the light chain of the anti-PDL1 antibody) were obtained by the two backbone plasmids formed above via the virus rescue system and the method as described in Example 1. Further, the optimal infection ratio of the virus expressing the heavy chain of the anti-PDL1 antibody to the virus expressing the light chain of the anti-PDL1 antibody was further determined by immunoblotting assay.

As shown by the experimental results in part A of FIG. 2, it was determined by Western blotting that the maximum amount of intact anti-PDL1 antibody secreted into the supernatant could be achieved when the ratio of the heavy chain of the anti-PDL1 antibody to the light chain of the anti-PDL1 antibody was 6:1 (i.e., the multiplicity of infection of RV-PDL1-H was 6 times of RV-PDL1-L). Similarly, the genes of the heavy chain and the light chain of the anti-PDL1 antibody were cloned into the backbone plasmid pRV-2MCS at the same time with reference to the cloning steps of pRV-PDL1-H and pRV-PDL1-L, so as to form a new cloning vector pRV-PDL1-(H+L). The specific sites to which the genes of the heavy chain and the light chain of the anti-PDL1 antibody were cloned were as shown in FIG. 1B. The gene of the heavy chain of the anti-PDL1 antibody was cloned to specific sites via XhoI and AscI, and the gene of the light chain of the anti-PDL1 antibody was cloned to the backbone vector via NheI and NotI. As shown in part B of FIG. 2, the recombinant virus RV-PDL1-(H+L) corresponding to the cloning vector pRV-PDL1-(H+L) replicated and expressed exogenous proteins (the heavy chain and the light chain of the anti-PDL1 antibody) in Vero cells, and the presence of a large amount of intact anti-PDL1 monoclonal antibodies was further detected in the secreted supernatant by immunoblotting assay, which proved that pRV-2MCS plasmid system integrated with both the gene of the heavy chain and the gene of the light chain of the antibody against the immune checkpoint was capable of efficiently expressing active and intact monoclonal antibodies in engineered Vero cells.

### Example 3: Stability of the Attenuated Virus Vector System pRV-2MCS

The gene sequences of GFP and RFP were simultaneously cloned into the attenuated virus vector system pRV-2MCS to form a new cloning vector pRV-2MCS-GFP-RFP. After the recombinant virus was rescued, the engineered Vero cells were co-incubated with the recombinant virus for 12 h, and then both green fluorescence and red fluorescence could be observed in most of the cells in a fixed field of view under a fluorescence microscope, as shown in part C of FIG. 2. The above-mentioned experimental results directly proved that both GFP fluorescent protein and RFP fluorescent protein were expressed by pRV-2MCS-GFP-RFP in cells.

Furthermore, after the recombinant pRV-2MCS-GFP-RFP was passaged five times, the corresponding detection of the expression of the exogenous genes showed no difference in expression efficiency when compared with the first generation infection, thereby further proving the stability and high efficiency of the pRV-2MCS rhabdovirus-based system.

### Example 4: Selection of Exogenous Anti-PDL1 Single-Chain Antibody Sequence Integrated into the Attenuated Virus Vector System pRV-2MCS

Based on the anti-PDL1 antibody known in the prior art, the anti-PDL1 amino acid sequence in the AVTM recombinant vector expressing exogenous anti-PDL1 single-chain antibody (RV-scFV-PDL1) was optimized.

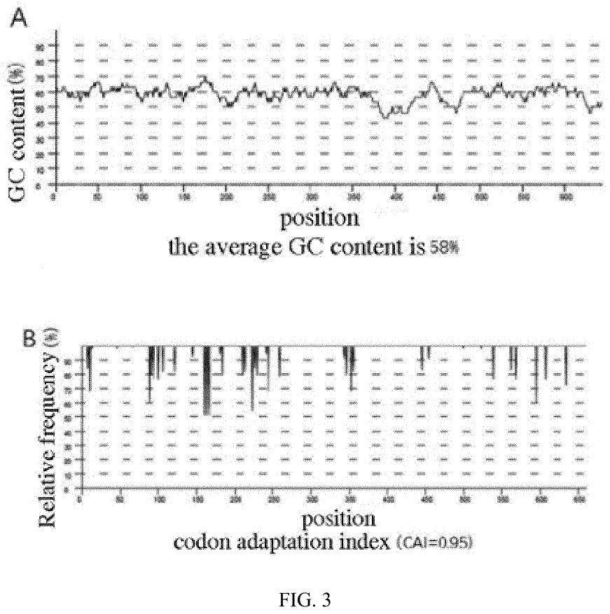

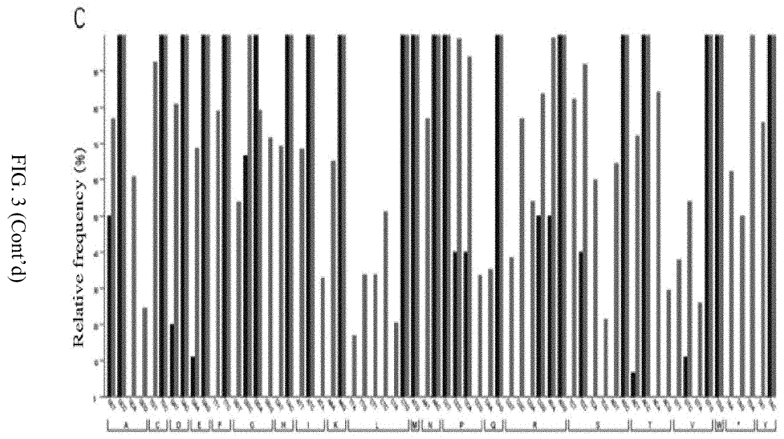

Experimental results: As shown in part A of FIG. 3, the average GC content in the designed and optimized gene sequence of the exogenous anti-PDL1 single-chain antibody was 58%. As shown in part B of FIG. 3, in the schematic diagram of codons, the multiplicity of codon preference was modified to the most reasonable range (CAI=0.95). Part C of FIG. 3 showed the optimal distribution pattern of a variety of codons in mammalian cells and the relative distribution frequency of the synonymous codons of amino acids in the gene sequence.

### Example 5: Characteristics of the Attenuated Rhabdovirus Strain RV-Mut4 Screened by the Attenuated Strain Screening System

Attenuated rhabdovirus strains with different point mutations, including RV-M51R (a single-mutant strain), RV-M51R-V221F (a double-mutant strain), RV-G21E-M51R-L111F (a triple-mutant strain), RV-G21E-M51A-L111F (a triple-mutant strain), RV-G21E-M51A-L111F-V221F (a quadruple-mutant strain, i.e., RV-Mut4) were obtained by screening according to the screening method described in Example 1 and the attenuated strain screening system as shown in FIG. 1A.

The specific method and steps used in this Example were as follows.

MTT experimental method:

1. 100 μl of LLC cell suspension was added to each well of a 96-well culture plate, so as to enable the cell density to reach 1×10⁴ cells/well, The cells were incubated at 37° C. in 5% CO₂ atmosphere for 16 h.

2. The viruses were respectively diluted to 10⁴ PFU, 10³ PFU, 10² PFU and 10¹ PFU, the diluted resultant of each gradient was inoculated into 4 wells (100 μl per well). The cells were incubated at 37° C. in 5% CO₂ atmosphere for 40 h.

3. The supernatant in the 96-well culture plate was discarded, fresh culture medium was added, and MTT solution was added (20 μL/well). The cells were incubated at 37° C. in 5% CO₂ atmosphere for 4 h.

4. The 96-well plate was centrifuged at room temperature for 5 minutes and the rotation speed was set to 2500 rpm/minute.

5. A 1-mL disposable sterile syringe was used to gently aspirate the supernatant.

6. DMSO was added to each well (100 μl/well), and the 96-well plate was left to stand at 37° C. for 10 minutes.

7. A multifunctional microplate reader was used to determine the OD value of each well at a wavelength of 570 nm or 490 nm after the 96-well plate was shaken for 2 minutes.

The total number of GFP-positive cells was determined by flow cytometry.

1. 100 μl of Vero (LLC/Hela/MEF) cell suspension was added to each well of a 48-well culture plate, so as to enable the cell density to reach 2×10⁴ cells/well. The cells were incubated at 37° C. in 5% CO₂ atmosphere for 16 h.
2. Specific mutant virus strains (100 PFU) were respectively added to each well, each kind of virus was added to 21 wells, and 12 wells were set as the blank control group.
3. Cells were respectively harvested at each time point (24 h, 36 h, 48 h, 60 h, 72 h, 84 h, and 96 h), the cells in three wells were harvested for each kind of virus and the cells in one well were harvested for the blank control group. The obtained cells were all re-suspended with 400 μl of PBS, 100 μL of the cell suspension was taken and analyzed by using Life Launch Attune NxT-Next flow cytometer, and the total number of GFP-positive cells was counted.

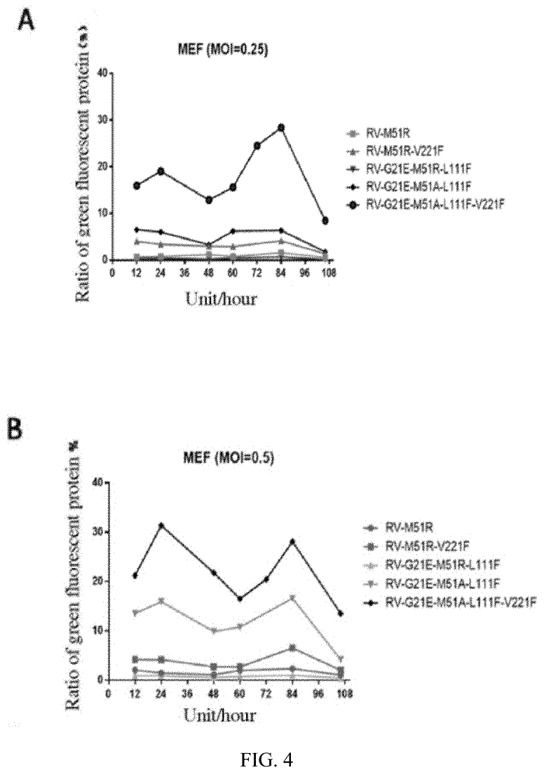

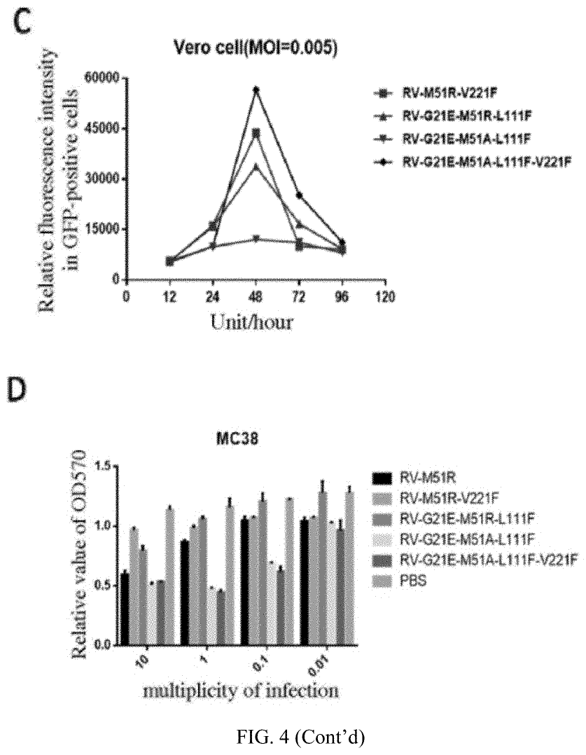

As shown in part A of FIG. 4 and part B of FIG. 4, 100 μl of MEF/Vero/MC38 cell suspension was first added to each well of a 48-well culture plate, so as to enable the cell density to reach 2×10⁴ cells/well. Specific mutant virus strains (100 PFU) were respectively added to each well, each kind of virus was added to 21 wells, and 12 wells were set as the blank control group. Cells were respectively harvested at each time point (24 h, 36 h, 48 h, 60 h, 72 h, 84 h, and 96 h), the cells in three wells were harvested for each kind of virus and the cells in one well were harvested for the blank control group. The obtained cells were all re-suspended with 400 μl of PBS, 100 μL of the cell suspension was taken and analyzed by using Life Launch Attune NxT-Next flow cytometer, and the total number of GFP-positive cells was counted.

Experimental results: As shown by part A to part C of FIG. 4, it was found that when the mutant strains obtained by screening (that is, RV-M51R (a single-mutant strain), RV-M51R-V221F (a double-mutant strain), RV-G21E-M51R-L111F (a triple-mutant strain), RV-G21E-M51A-L111F (a triple-mutant strain) and a quadruple-mutant strain RV-Mut4 (RV-G21E-M51A-L111F-V221F)) were subjected to a comparison on the expression of exogenous genes, RV-Mut4 had the ability to continuously express the exogenous protein in cells, and the expression level reached a peak after three days and then gradually decreased. It was found by further comparison experiments that, as compared with the mutant strain in control groups, the expression level of the exogenous gene expressed by RV-Mut4 reached a peak at 48 h and was much higher than those of the control groups (as shown in part C of FIG. 4). The expression level of the exogenous protein (GFP) that had been integrated into RV-Mut4 attenuated strain increased significantly, while the specific killing effect of the virus itself on tumor cells was not weaken. As shown in part D of FIG. 4, the ability to lyse and kill tumor cells showed no significant difference from the control groups.

In summary, among the above-mentioned virus strains, RV-Mut4 had the most prominent characteristics. The four amino acid mutations in this strain did not make the virus more virulent and retained the specificity in killing tumor cells at the same time. Meanwhile, although the time point when tumor cells were lysed was found to be delayed at the cellular level in vitro, the ability to specifically kill tumor cells was completely retained. Further, RV-Mut4 showed no toxicity to normal cells, which fully conformed to the requirements for biosafety.

### Example 6: Expression of RV-scFV-PDL1 Vector in Vero Cells

Experimental procedures: By using the pRV-2MCS system as shown in FIG. 2 and the method as shown in Example 1, based on the pRV-2MCS blank backbone plasmid, the scFV-PDL1 gene was cloned to the backbone plasmid pRV-2MCS via a double enzyme digestion system comprising specific endonucleases XhoI and NheI, wherein the exogenous scFV-PDL1 gene was located between G gene and L gene on the core backbone of the virus (for detailed steps, please refer to the process of cloning an exogenous gene into a core backbone plasmid as described in Example 1). A new backbone plasmid was obtained and named pRV-scFV-PDL1. Further, the recombinant virus RV-scFV-PDL1 was rescued and obtained in BSR-T7 cells, and the supernatant containing the products generated by the recombinant virus in Vero cells was collected. After the supernatant was respectively co-incubated with (human) MC-38-hPDL1 cells and LLC-hPDL1 cells which highly expressed CD274 on cell surface at room temperature for one hour (the anti-PDL1 single-chain antibody expressed and secreted in the supernatant was capable of binding to CD274 which was a receptor molecule highly expressed on cell surface), the specific amount and proportion of anti-PDL1 antibody-positive cells were determined by flow cytometer.

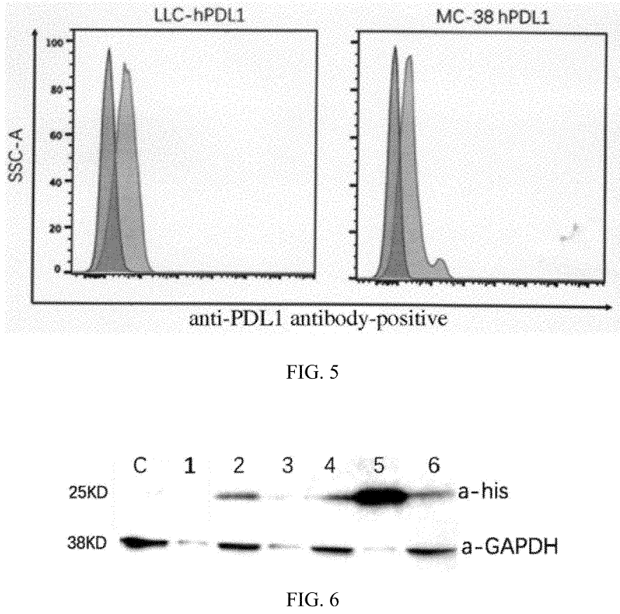

Experimental results: As shown in FIG. 5, the proportion of anti-PDL1 antibody-positive cells exceeded 50%, indicating that RV-scFV-PDL1 was capable of expressing active anti-CD274 single-chain antibodies via cells. Meanwhile, such results further proved that the single-chain antibody expressed by RV-scFV-PDL1 had the ability to bind human CD274 and murine CD274 and had human/mouse cross-reactivity, which were beneficial for the rapid verification of drug efficacy in preclinical animal models.

### Example 7: Effect of Signal Peptides on RV-scFV-PDL1

By chimerizing the variable regions of the heavy chain and light chain of the exogenous immunoglobulin in an established non-truncated negative-strand RNA vector system AVTM, a specific linker sequence (15 amino acids) for the variable regions of the heavy chain and the light chain was designed, that is, internationally accepted (G4S)₃ was used as a flexible polypeptide linker, and four different signal peptides for secretory antibody were designed at the same time. The effect of each signal peptide on the secretion efficiency of the single-chain antibodies was compared with other signal peptides at cellular level in vitro (293T cells were subjected to transient transfection with the plasmid which was then expressed in the system).

The specific experimental procedures of the above-mentioned experiment were as follows.

1. Four signal peptide sequences (Sig1, Sig2, Sig3 and Sig4, please refer to Table 2 for their sequences, four base sequences in total) were added at the N-terminal of the sequence of the anti-PDL1 single-chain antibody, respectively. These four gene sequences were respectively cloned into the pcDNA3.1 backbone (purchased from ATCC Co.) by utilizing molecular cloning technique, so as to finally form four eukaryotic expression vectors, that is, pcDNA3.1-sig1-scFV-PDL1, pcDNA3.1-sig2-scFV-PDL1, pcDNA3.1-Sig3-scFV-PDL1, and pcDNA3.1-Sig4-scFV-PDL1.
2. After 293T cells were transfected with the above four vectors for 48 h, the supernatant containing the secreted products and cells were collected respectively. Samples were prepared by the lysis of cells with a loading buffer, and the expression level of the anti-PDL1 single-chain antibody in the supernatant was determined by immunoblotting technique (pcDNA3.1 vector carried a His tag, and the expression of the target protein was detected by an anti-His tag antibody).

TABLE 2
Amino acid sequences of signal peptides
for secretory antibody

| | | |
|---|---|---|
| Sig1 | signal peptide | MLLTLIILLPVVSK |
| Sig2 | signal peptide | MWLQSLLLLGTVACSIS |
| Sig3 | signal peptide | MYRMQLLSCIALSLALVTNS |
| Sig4 | signal peptide | METDTLLLWVLLLWVPGSTG |

The experimental results were as shown in FIG. 6. 293T cells were transfected with eukaryotic expression vectors that were respectively linked to three different signal peptides. Cells and supernatant medium were collected separately. A codon-optimized scFV-PDL1 was synthesized and a His tag was incorporated at the 3′-end. The secretion and expression level of the antibody by the expression vectors in cells were determined by immunoblotting assay and said expression vectors were respectively linked to four different signal peptides. It was found that, as compared with the other three signal peptides, the anti-PDL1 scFV antibody linked to Sig3 signal peptide had the highest secretion efficiency and the expression level showed an exponential increasing trend.

Four signal peptides for antibody secretion as mentioned in Table 2 were respectively integrated into the pRV-core Mut4 plasmid system via molecular cloning technique. The rescue process of the recombinant virus was completed in accordance with the following virus rescue technique. Vero E6 cell line was infected with four recombinant viruses with a multiplicity of infection (MOI) of 0.01. Cell supernatant was collected 24 hours later, and the expression level of the single-chain antibody in the supernatant was detected.

The specific steps of the above-mentioned virus rescue technique were as follows. 5×10⁶ BSR-T7 cells were evenly seeded in a 10-cm dish and cultured overnight with DMEM supplemented with 10% fetal bovine serum until the cell density reached 80%. Cells were washed with serum-free DMEM twice one hour before transfection. Using a calcium phosphate transfection kit, cells were transfected with 5 μg of the backbone plasmid pRV-core Mut4 (or a recombinant core backbone plasmid), 5 μg of pN (a plasmid expressing the nucleoprotein of the rhabdovirus), 2.5 μg of pL (a plasmid expressing the polymerase protein of the rhabdovirus), and 2.5 μg of pP (a plasmid expressing the phosphoprotein of the rhabdovirus). After 2 h to 3 h, cells were washed twice with PBS and cultured in DMEM supplemented with 10% fetal bovine serum for 3 days. The resulting cell supernatant were co-incubated with Vero cells at 37° C. for 3 days. The expression of the viruses rescued in BSR-T7p cells was determined by immunofluorescence assay with an FITC-labeled rhabdovirus G protein-specific antibody.

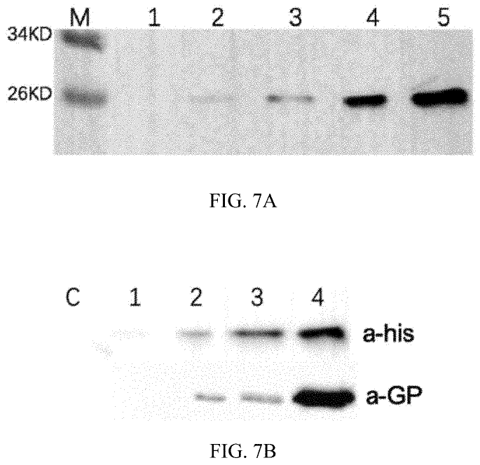

The results of virus rescue were as shown in FIG. 7A. FIG. 7A indicated that the expression system comprising sig3 or sig4 showed rather high expression level in the supernatant while the expression level of the expression system comprising sig1 or sig2 was almost undetectable.

Further, the attenuated strain RV-scFV-PDL1 (sig3) expressing sig3 as the signal peptide was inoculated via subcutaneous injection adjacent to the tumor site and intratumoral injection. Two days after the inoculation, the serum and local tumor tissues of the model mice were respectively collected. Tissue samples were ground, and the expression of the antibody and the presence of virus-related proteins at different sites of the tumor-bearing mouse model were respectively determined by immunoblotting.

The experimental results were as shown in FIG. 7B. As compared with the group in which the mice were not inoculated with the virus (Lane C), after the attenuated strain RV-scFV-PDL1 (sig3) was administered via subcutaneous injection adjacent to the tumor site, the presence of viral envelope protein was detected in mouse tumor tissue (Lane 2) and the expression of the single-chain antibody expressed by the exogenous gene was also detected at the same time, while the expression of virus-related genes was not detected in the serum of corresponding mice (Lane 1). Further, mice were administered intratumorally and topically. Significant presence of virus-related proteins was detected in both serum (Lane 3) and tumor tissues (Lane 4). In particular, when the mice were administered intratumorally, the expression level of the exogenous protein in tumor tissues increased significantly. The experimental results directly proved that RV-scFV-PDL1 (sig3) had the ability to express the exogenous single-chain antibody rapidly and efficiently in tumor tissues.

### Example 8: Test and Evaluation of the Immunotherapy Efficacy of RV-scFV-PDL1 in Metastatic Non-Small Cell Lung Cancer Model

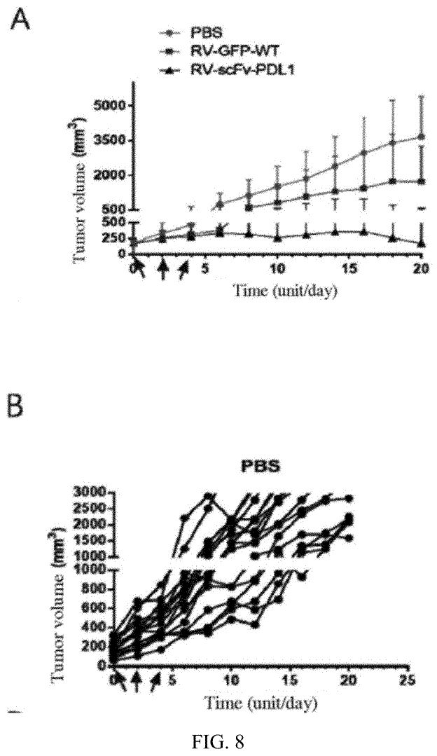

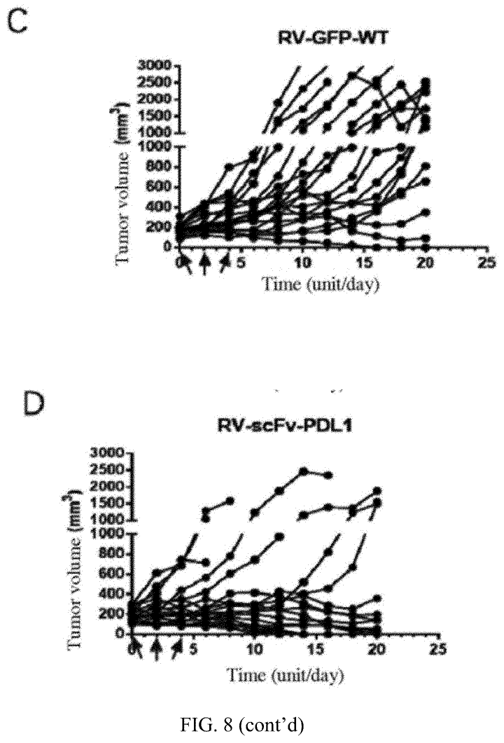

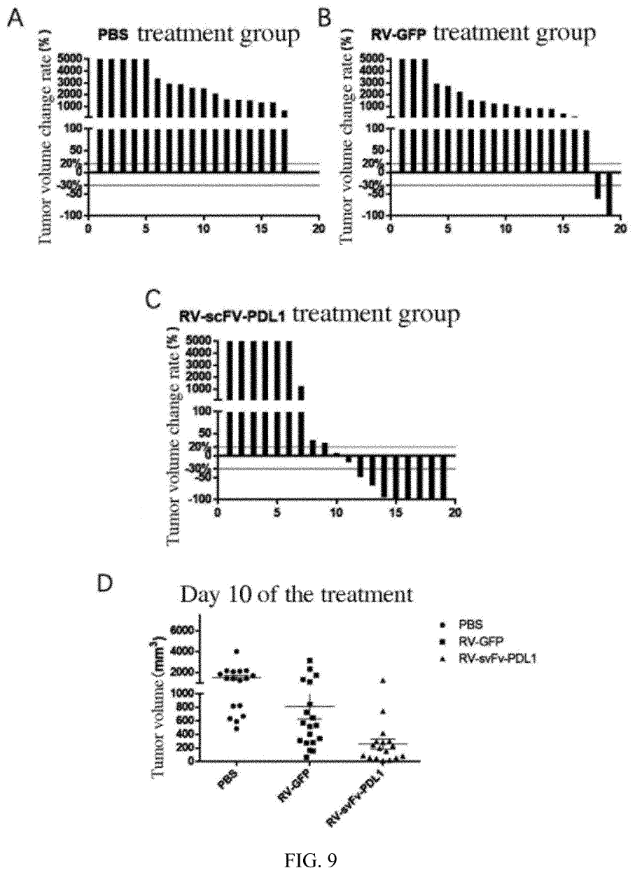

A metastatic non-small cell lung cancer model was first established. As shown in part A of FIG. 8, each C57BL/6 mouse was subcutaneously inoculated with 1.0×10⁶ (200 μL) LLC-JSP cells (purchased from ATCC, USA). Tumor sizes were measured every other day and tumor volumes were calculated according to the following formula: M1²×M2/2 (M1: short diameter, M2: long diameter). After the tumor volumes of the model mice in each group reached approximately 200 mm³, 10⁶ PFU (20 μl) of the virus was administered via intratumoral injection for treatment on day 12, day 14 and day 16, respectively. Changes in tumor volume were continuously observed and recorded.

Experimental results: As shown in part A of FIG. 8, there were three treatment groups, namely, AVTM system-mediated single-chain antibody group (the attenuated strain RV-scFV-PDL1), RV-WT experimental control group and PBS blank control group. Part A of FIG. 8 illustrated the overall statistical diagram showing the therapeutic effect on solid tumor in LLC non-small cell lung cancer model (mouse model). In order to further determine the inter-individual difference among different treatment groups, individuals in each group were subjected to statistical analysis separately. Part B of FIG. 8 was an individual statistical diagram of PBS treatment group. Part C of FIG. 8 was a schematic individual statistical diagram of RV-GFP-WT treatment group. Part D of FIG. 8 represented an individual statistical diagram of RV-scFV-PDL1 treatment group. Model mice were inoculated intratumorally (i.e., in the tumor tissues of non-small cell lung cancer models) with 10⁷ PFU (30 μl) of the attenuated virus every other day (three times in total). Tumor volume changes of the model mice in each experimental group were measured every other day. Part D of FIG. 8 illustrated the test and evaluation of the immunotherapy efficacy of RV-scFV-PDL1. Three consecutive intratumoral injections administered every other day was capable of effectively inhibiting the growth trend of tumors and greatly prolonging the life span of the model mice. Based on the independent analysis of the therapeutic effect of RV-scFV-PDL1 system on each model mice receiving the treatment of this RV-scFV-PDL1 system, it was found that the tumors in approximately 40% of the model mice shrunk and finally disappeared (that is, complete response) and the tumor growth rate in approximately 30% of the model mice was effectively controlled (that is, partial response), with a overall response rate of approximately 70%. As compared with RV-GFP-WT group (control treatment group), the therapeutic effect was significant improved and the superiority was apparent.

Part A of FIG. 9 to part C of FIG. 9 further showed the tumor volume changes of each individual model mice in different treatment groups after 20 days of treatment. Furthermore, tumor volume changes of mice in different treatment groups on Day 10 (part D of FIG. 9) and Day 20 (part E of FIG. 9) of the treatment were analyzed. It could be found on Day 10 of the treatment that the development of non-small cell lung cancer was suppressed to a certain extent in the early treatment stage in RV-GFP experimental control group. When the observation period was prolonged to 20 days, the continuous shrinking of tumor could only be observed in two mice (n=19). However, in RV-scFV-PDL1 treatment group, a trend of continuous shrinkage in tumor volume was maintained from Day 10 to Day 20 of the treatment and the effective control rate was close to 80%, thereby proving that in RV-scFV-PDL1 intratumoral administration group, immune cells in the local region of tumor were activated by three doses, continuous anti-tumor immune response was induced, specific immune cells accumulated in the local region of tumor, tumor cells were eventually eliminated, and the tumor tissue shrunk and finally disappeared. It could be seen from the further analysis of the experimental results that, after RV-scFV-PDL1 was administered intratumorally three times, none of the model mice developed drug resistance and model mice showing therapeutic effect in initial stage did not suffer from recurrence in later period.

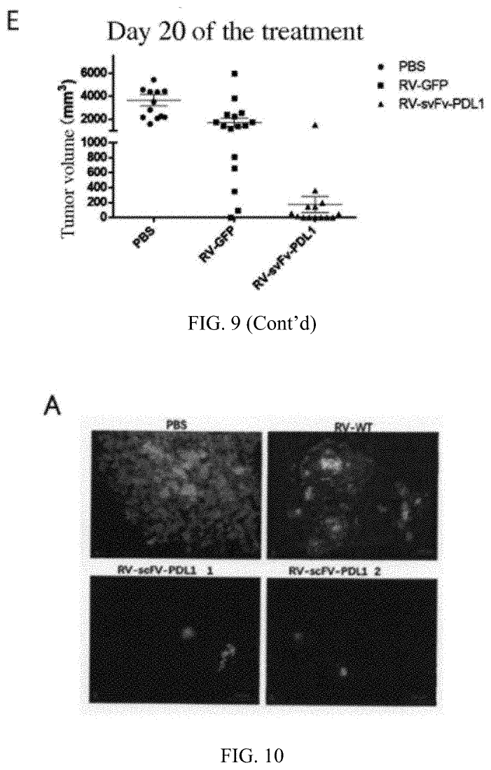

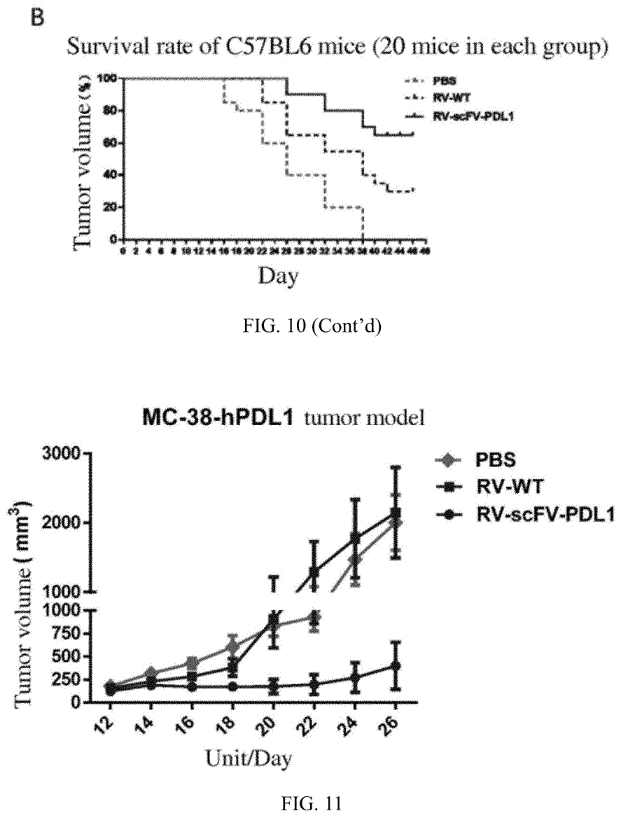

Further, the lung tissues of the model mice in the experimental group and the control groups were incised, and the metastasis of cancer cells (LLC-JSP) in the lung tissues (cancer cells that were subcutaneously inoculated and appeared in lung tissues due to metastasis) of the model mice was observed under a fluorescence microscope and recorded. It could be seen from part A of FIG. 10 that the model mice in RV-scFV-PDL1 immunotherapy group showed the fewest lung metastases. By recording the valid survival period of the tumor-bearing model mice that had received treatment, it was further found that the survival rate of the mice in the experimental group was the highest and approximately 65% of the model mice maintained a normal life state in nearly two months (as shown in part B of FIG. 10).

### Example 9: Test and Evaluation of the Immunotherapy Efficacy of RV-scFV-PDL1 in Colon Cancer Model

A tumor cell line expressing human PDL1 (MC38-hPDL1) was first established. Cells were inoculated to C57BL/6 mice to establish a humanized colon cancer mouse model. The efficacy of RV-scFV-PDL1 enabling the chimeric expression of a single-chain antibody universal for human and mouse was further verified.

The establishment of this colon cancer model was based on the knockout of murine PDL1 gene in colon cancer cell line (MC-38) and the subsequent chimeric expression of human PDL1. The steps and method for establishing the model were shown as below.

Step 1: Murine PDL1 (mPDL1) gene was knocked out by CRISPR-Cas9 method. According to CRISPR-Cas9 technique, a short guide RNA (sgRNA) was first designed and the designed sequence was as follows: 5′-GCTTGCGTTAGTGGTGTACT-3′. The synthesized double-strand DNA was inserted into the sgRNA expression vector (FG-BB-U6-sgRNA) via BbsI site. Then, MC-38 cells were co-transfected with the resulting vector and a Cas9-expressing plasmid (FG-hEF/HTLV-Cas9-PGK-Puro-WPRE). After 60 hours, a screening with puromycin (puro) lasted for 48 hours (for the expression vector, the transfection method and the screening method mentioned herein, please refer to Scientific Reports, Volume 7, Article number: 42687, Anfei Huang et al., Feb. 16, 2017).

Afterwards, the culture was expanded to obtain an mPDL1 gene-knockout cell bank, DNA was extracted, and PCR was performed with the following primers: forward primer: 5′-TGGTTCCTTTTAAACAAGACTGGG-3′, reverse primer: 5′-CGCACCACCGTAGCTGATTA-3′. PCR products were collected and subjected to TA cloning, and the resultants were then sent for sequencing.

Step 2: A lentiviral system was used to enable the overexpression of human PDL1 in such mPDL1 KO MC-38 cells. Human PDL1 gene was first cloned and inserted into the adenovirus expression vector FG-hEF/HTLV-human CD274-PGK-Puro-WPRE, and HEK293T cells were then transfected with the vector to enable virus packaging. MC-38 mPDL1 KO cells were infected with the resulting virus for 48 h, and a screening with puromycin lasted for 48 hours.

The knockout efficiency of mPDL1 was determined by sequencing method. Interferon-γ (IFN-γ) was capable of stimulating the expression of PDL1 significantly. The expression levels of PDL1 in normal cells and mPDL1 gene-knockout cells were respectively determined by flow cytometry before and after IFN-γ stimulation, and the results further indicated the successful knockout of mPDL1 gene in MC-38 cells.

Similarly, the expression level of hPDL1 (overexpressed by lentivirus) in mPDL1 KO MC-38 cells was determined by flow cytometry. The results of the flow cytometry indicated that hPDL1 could be expressed normally in this cell line.

In summary, a tumor cell line expressing human PDL1 (MC-38-hPDL1) was established after the verification of each step. By recording the valid survival period of the tumor-bearing model mice that had received treatment, it was further found that the survival rate of the model mice in RV-scFV-PDL1 treatment group was the highest and approximately 70% of the model mice maintained a normal life state in nearly two months (as shown in FIG. 11). In the efficacy evaluation conducted in colon cancer mouse model, as shown in FIG. 11, after intratumoral administration given every other day (three times in total), tumor growth was significantly inhibited and the life span of the model mice was prolonged in the treatment group as compared with the control group (PBS group). According to the single-individual analysis of the treatment group, tumor volume decreased continuously or tumor volume was maintained at a certain volume in approximately 70% of the individuals. As compared with the control group, the increase of tumor volume was significantly controlled in RV-scFV-PDL1 treatment group with remarkable effect and excellent therapeutic efficacy. Meanwhile, mediating the expression of the antibodies targeting immune checkpoint(s) by utilizing AVTM virus vector system was feasible for the clinical cure of such cancers. AVTM vector system had reliable targeting ability for the potential treatment of malignant tumors and was less likely to induce drug resistance. AVTM vector system could be administered repeatedly for multiple times with remarkable therapeutic efficacy.

The above-mentioned Examples of the present disclosure are merely exemplified to clearly illustrate the present disclosure rather than limitations to the embodiments of the present disclosure. For those of ordinary skill in the art, other changes or modifications in different forms may also be made based on the foregoing description. It is not necessary and impossible to enumerate all the embodiments. Any modification, equivalent replacement and improvement made within the spirits and principles of this disclosure shall be encompassed in the protection scope of the claims of the present disclosure.

1. A preparation method of an attenuated virus vector, comprising the following steps:
(S1) mixing a nucleotide sequence of a gene encoding a matrix protein of a vesicular stomatitis virus as set forth in SEQ ID NO:1 (M gene of VSV) with a first vector, and adding a transposase to initiate a transposition reaction;
(S2) mixing a transposition product obtained in step (S1) with a competent bacterium for transformation;
(S3) extracting a plasmid of a bacterium obtained by step (S2) so as to obtain a transpositioned gene encoding the matrix protein of the vesicular stomatitis virus; and
(S4) recombining the gene obtained in step (S3) into a second vector so as to obtain the attenuated virus vector;
wherein a sequence encoding the second vector comprises a genome sequence of the vesicular stomatitis virus;
wherein the first vector is a vector with transposition function;
alternatively, the first vector comprises a sequence as set forth in SEQ ID NO:2;
alternatively, the second vector comprises a sequence as set forth by a sequence numbered EU849003.1 in GENEBANK.

2. An attenuated virus vector obtained by the preparation method of claim 1.

3. (canceled)

4. The attenuated virus vector according to claim 2, wherein a sequence encoding the attenuated virus vector comprises a sequence as set forth in SEQ ID NO: 4.

5. A cloning backbone vector system, wherein a sequence as set forth in SEQ ID NO:5 is recombined into the vector of claim 4 in the cloning backbone vector system; wherein a position where the sequence as set forth in SEQ ID NO:5 is inserted into a sequence encoding the vector of claim 4 is position 4632 in a sequence as set forth in SEQ ID NO: 4.

6. The cloning backbone vector system according to claim 5, wherein a sequence encoding the cloning backbone vector system comprises a sequence as set forth in SEQ ID NO:6.

7. A method for preparing an attenuated monoclonal virus strain, comprising the following steps:
(S1) culturing a first cell to be transfected;
(S2) co-transfecting the cell to be transfected in step (S1) with a plasmid mixture of a plasmid comprising a sequence as set forth in SEQ ID NO:3, a plasmid comprising a sequence as set forth in SEQ ID NO:7 (pN), a plasmid comprising a sequence as set forth in SEQ ID NO:8 (pL) and a plasmid comprising a sequence as set forth in SEQ ID NO:9 (pP);
(S3) extracting a supernatant of a cell mixture obtained after co-transfection in step (S2) and transfecting a second cell to be transfected with the supernatant; and
(S4) culturing and screening the second cell to be transfected that has been transfected in step (S3), so as to obtain the attenuated monoclonal virus strain.

8. The method according to claim 7, wherein the plasmid comprising the sequence as set forth in SEQ ID NO: 3-4, the plasmid comprising the sequence as set forth in SEQ ID NO:7 (pN), the plasmid comprising the sequence as set forth in SEQ ID NO:8 (pL) and the plasmid comprising the sequence as set forth in SEQ ID NO:9 (pP) have a weight ratio of 10:4:1:5.

9. (canceled)

10. The method according to claim 8, wherein the plasmid comprising the sequence as set forth in SEQ ID NO:4 in step (S2) is a plasmid comprising a sequence as set forth in SEQ ID NO:6.

11. The method according to claim 10, wherein an encoding sequence of the plasmid comprising the sequence as set forth in SEQ ID NO:6 in step (S2) further comprises a sequence as set forth in SEQ ID NO:10 and a sequence as set forth in SEQ ID NO:11, alternatively, wherein an encoding sequence of the plasmid comprising the sequence as set forth in SEQ ID NO:6 in step (S2) further comprises a sequence as set forth in SEQ ID NO:12.

12. (canceled)

13. The method according to claim 11, wherein a sequence comprising the sequence as set forth in SEQ ID NO:12 further comprises a signal peptide sequence at 5′-end; the signal peptide sequence is selected from sequences as set forth in SEQ ID NO:13, SEQ ID NO:14, SEQ ID NO:15 and SEQ ID NO:16; preferably, the signal peptide sequence is a sequence as set forth in SEQ ID NO:15.

14. An attenuated monoclonal virus strain prepared and obtained by the method for preparing an attenuated monoclonal virus strain according to claim 7.

15. A monoclonal antibody secreted by the attenuated monoclonal virus strain according to claim 14.

16. A monoclonal antibody comprising fragments encoded by encoding sequences of a sequence as set forth in SEQ ID NO: 6, a sequence as set forth in SEQ ID NO:10 and a sequence as set forth in SEQ ID NO:11.

17. (canceled)

18. The monoclonal antibody according to claim 16, wherein the encoding sequence further comprises a nucleotide sequence encoding a sequence as set forth in SEQ ID NO:13, SEQ ID NO:14, SEQ ID NO:15 or SEQ ID NO:16; preferably, the encoding sequence further comprises a nucleotide sequence encoding the sequence as set forth in SEQ ID NO:15.

19. A pharmaceutical composition comprising the monoclonal antibody of claim 15.

20. (canceled)

21. (canceled)

22. (canceled)

23. A method for slowly and continuously killing abnormally proliferating cells, comprising a step of contacting the abnormally proliferating cells with the monoclonal antibody according to claim 15.

24. The method according to claim 23, wherein the abnormally proliferating cells are contained in the body of a patient.

25. The method according to claim 23, wherein the abnormally proliferating cells are selected from tumor cells or tumor tissue-related cells; preferably, the tumor cells are cancer cells; and more preferably, the cancer cells are metastatic cancer cells.

26. (canceled)

27. (canceled)

28. The method according to claim 23, wherein the method further comprises a step of administering a second antitumor therapy.

29. (canceled)

30. A polynucleotide comprising a sequence as set forth in SEQ ID NO:6.
